SEQUENCE LISTING (22 sequences)

SEQ ID NO: 1 - protein, 1499 aa, Legionella pneumophila
  SdeA
MPKYVEGVELTQEGMHAIFARMGYGDITSGSIYNGVPTIDTGALNRQGFMPVLTGVGPHRDSGHWIMLIKGPGNQYYLFDPLGKTSGEGYQNILAAQLPMGSTLSVIPNGSGLNMGLCGYWVASAGLRAHQALNQHNPPTLLNVGQTITNEMRNELDHDGYRKITGWLRAVADEFPEGDPQLDGKALRENTEKDLKIEIPTLVLPGKDTSPKEMSVKPTAPQDKSVPVWNGFSLYTDDTVKAAAQYAYDNYLGKPYTGSVESAPANFGGRMVYRQHHGLSHTLRTMAYAELIVEEARKAKLRGETLGKFKDGRTIADVTPQELKKIMIAQAFFVAGRDDEASDAKNYQKYHEQSRDAFLKYVKDNESTLIPDVFKDQEDVNFYARVIEDKSHDWESTPAHVLINQGHMVDLVRVKQPPESFLQRYFSSMQRWIGSQATEAVFGIQRQFFHATYEVVAGFDSDNKEPHLVVSGLGRYVIGEDGQPIREAPKKGQKEGDLKVFPQTYKLKENERLMRVDEFLKLPEIQNTFPGSGKHLQGGMPGMNEMDYWNRLNSLNRARCENDVDFCLKQLQTAHDKAKIEPIKQAFQSSKGKERRQPNVDEIAAARIIQQILANPDCIHDDHVLINGQKLEQQFFRDLLAKCEMAVVGSLLNDTDIGNIDTLMRHEKDTEFHSTNPEAVPVKIGEYWINDQRINNSSGNITQKKHDLIFLMQNDAWYFSRVNAIAQNRDKGSTFKEVLITTLMTPLTSKALVDTSQAKPPTRLFRGLNLSEEFTKGLIDQANAMIANTTERLFTDHSPEAFKQIKLNDLSKMSGRTNASTTTEIKLVKETWDSNVIFEMLDPDGLLHSKQVGRHGEGTESEFSVYLPEDVALVPVKVTLDGKTQKGENRYVFTFVAVKSPDFTPRHESGYAVEPFLRMQAAKLAEVKSSIEKAQRAPDLETIFNLQNEVEAVQYSHLSTGYKNFLKNTVGPVLENSLSGLMESDTDTLSKALAAFPSDTQWSAFNFEEARQAKRQMDAIKQMVGNKVVLDALTQCQDALEKQNIAGALDALKKIPSEKEMGTIRRELREQIQSARQELESLQRAVVTPVVTDEKKVRERYDALIENTSKKITELETGKLPNLDAVKKGISNLSNLKQEVTVLRNEKIRMHVGTDKVDFSDVEKLEQQIQVIDTKLADAYLLEVTKQISALDNTKPKNQTELKTKIAAFLDRTTDIEMLRNERIKKHGSSKDPLDLSDLDKLSGSLQRINQSLVSDLITTIRVSINQMEAKTFHEQEKEIQQNFELLAKLEKTLDKSKTSEKLREDIPKLNDLLVAKQKAYPQMVQMQLKSEVFVTQLREVCQANHDDLDKTRNARLRELDRLDREAGITRMVGNLIWGLTNKVGLTTDERLDIRTKQQSLARFKNELFNDKIDTDQLISNLARKRPSELQEGLGISTDNAMELHLLLTELAGKTTSPDELEERMKAIDDISTKIGREPEHLKFVMVEEDESNKKTIGF

SEQ ID NO: 2 - protein, 1926 aa, Legionella pneumophila
  SdeB
MGESKMPKYVEGVELTQEGMHAIFARMGHPEIKSGTIYNGVPTIDQEALDKQGFMPVLTGVGPKRDSGHWIMLIKGSGNQYHLFDPMGKISGEGYQDILATQLPKGSTLSVIPNEPGLNKGLCGYWVASVGLKACSALNKDNPPNLETLGQITTDAMKDELTDNGYLKITGWLKAVADKFPEGDPQPDAKALRQTTEKDLHIEIPSPVSPVKDTAPKEVSTKPTAPQVAPKYSLDSKLLENDDDVLDTIKYVHKEYLGKPYPGPLKNPKAPEEGRLPPNEGPDRGPHGLAHTVRTMACAEVMIEEARKAQLRGETLGKAKNGQTLADVTPEELKKILIAQAFFVVGRDDERSGYDDVHKRNFYAEYHEKSEQAFRKYVEDNKLIGKIFKDQKEVDFYAAIILDKNHEWDATPAHILINQGHMVDLMRTKAPAEVALERTYNTLKGTVGSKGAEVVLKAHRDFFFATGAVVPLVNPEAIDDPSRGGPYENPYSGEKFVIVDDKVPASKKDLPKAVNRDYKLKDNERFLTIKEYYAFPDVQQTYPGYKTRLEASSYYFPTPFAGECEQNPAKCLGAIQKARSKLQTDAIKNGFQSSSEKERRQPNMDEIAAARIIQQIMANPDCIHDDHVLINGQKLEEKFFRDLLAKCDMAVVGSLLNDTDIKNIDTLMRHEKNTEFHSTDPKAVPVKIGDAWENRIRTKGGDVTQMKHDLIFLMQNDAWYFSRVNAIAQNRDKGSNFKEVLFTTLMTPLTNKSLIDTSHVPAPKKLYRGLNLPQEFTNKLINQSNAIIANTENTLFTDLSAEAFKQIKLNDFSQMSGKTCASTTKNMKLLTDIWGSNVIFEMLDPDGLLHPKQVGTHMTGSEDEFSVYLPEDVALVPTKVTLEGKTDTGEDRYIFTLVAVKSPDFIPRHESGYAVEPFMKMQKEKVTQALDAIEKDKDSYNIDEQLKSLRTEMVRQAKLPLREGVFDRLSHRFSLETSDNKISPERRDFLNQHVIPVLQECHIALRTNNMEMMQKALAKFPTDKQWSAFKSGEAVRAKAQMDVLKQQIEKKIMLQTQIIPALTECGEALDKQNVTEALQALNKLPAEKEIGKAKGIGQELRGQIVGVKQELTGNLESLQRAVTTPVVKDAEKMRVRYETLLTDVTKRVTDFEKIKPANLDGYNKAIADLNNIQQELNLLRNEKIRMHTDKDKAVDFSDIEALEKRLKEAQPNLYPSLVEGTTKGIQELEKIPKSISFDDVKSMTSKINGYLETLEFIRNERVKKHGESTEPLDMSDLDKLKNQLQGVSQHLAQILLNGAKNSLDKIKDPVTLEKEGQYIKRCLDHFAELEKTLDSSVKGMKQKEDFNTYKNSLMEKQEKAYPEMLQLQYKSEALITQLRNLCNIHHDNLAKTREEQQQLLNKTGGILGGFFSQITTAISQAESKKANEFARFKTELNNDKSDVSQLIQFLVKKKPSELEENLGISKENAEQLHGLLKQLDAKATPIDKLQENTRLIDEISTKMGSKPVSPEHVAATKAREYYQFLLYGATQKIGNFEKIKPIDPESYKKAMSDLNNMQEALQFLRSEKSRIHDGKEKAVEFSDIEALEKRVQNAQPKLLTALLDKTTREISALVKIPRKLTFEDIKSMTTKLNSYLETLELIRNERIKQHGKSPELLDMSDLDSLKGQLQTFNQNLTGMILNATKDGLDRINDRANFKEGEPYVKACLDLLTELEKTLDSSVKGMKQKEDISACRNSLLDKQEKANSGMLDLQSKSKDLVTQLRDICKIHHGNLAEARRTRLHSLDNQEGGLLGGLWSVTNKLGVTTDTVGIERMQIKMKEQALARFKTELNNDKYDTNQVIAFLANKKPSELEEGLGISKENAEELHGLLSKLTSKMTSKIEIEENTQLIDEISTKIGTEPVKLESTHTVDEDERDTYHRESGYF

SEQ ID NO: 3 - protein, 1534 aa, Legionella pneumophila
  SdeC
MPKYVEGVELTQEGMHAIFARMGHGDITSGSIYNGVPTIDTEALNRQGFMPILTGVGPKRDSGHWIMLIKGPGNQYFLFDPLGKTSGEGYKNTLLAQLPIASTLSVIPNDPGLNRGLCGYWVASVGLKARAELNKDNPPNLETLGQITTDAMKDELTDNGYPKITGWLKAVADKFPEGDPQPDAKALRQTTEKDLHIELPSPVSSVKDTAPKEVSTKPTAPQVALKHSLDSKLLENDEDVLDTIKYVHKEYLGKPYPGPLKNPKAPEEGRLPPSEGPDRGPHGLAHTLRTMACAEVMIEEARKAKLRGETLGKAQNGQTLADVTPEELKKILIAQAFFVVGRDDERSGYDDVHKRNFYAEYHEQSEQAFRKYVEDNKLIGKIFKDQKEVDFYAAIILDKNHEWDATPAHILINQGHMVDLMRTKAPAEVALERTYNTLKYTLGSKGAEVVLKAHRDFFFATGAVVPLVNPEAIDDPSRGGPYENPFNGEKYVIVEGKEPKSTKDLPKPVGRNYQLKDNERFLTIKEYYAFPDVQQTYPGYKTRLEGSSYYFPTPFAGECEQNPAKCLGAIQKARSKLQTDAIKNGFQSSSDKERRQPNMDEIAAARIIQQIMANPDCIHDDHVSINGQKLEEKFFRDLLAKCDMAVVGSLLNDTDVKNIDTLMRHEKDTEFHSTDPQAVPVKIGDAWENRIRTKGGNVTQMKHDLIFLMQNDAWYFSRVNAIAQNRDKGSNFKEVLFTALMTPLTNKSLMDTSHVPAPKKLYRGLNLPQEFTNKLINQSNVIIANTENTLFTDLSAETFKQIKLNDFSQMSGKTCASTTKNMKLLTDIWGSNVIFEMLDPDGLLHPKQVGTHMTGSEDEFSVYLPEDVALVPTKVTLEGKTDTGEDRYIFTLVAVKSPDFIPRHESGYAVEPFMKMQKEKVTQALDAIEKEKGSYNIDEQLKNLRTEMVRQAKLPLREGVFDRLSHRLSLETSDNKISPERRDFLNQHVIPVLQECHIALRTNDMEMMQNALAKFPTDKQWSAFKSGEAVRAKAQMDVLKQQIEKTIMLQSQIIPALTECGEALDKQNATEALQALNKLPTEKEIAKAKGIGQELRGQITGVKQELTGNLEPLQRAVTTPVVKDAEKMRVRYETLVTDVTKRVTDFEKIKPANLESYNKAIADLNNIQQELNLLRNEKIRMHTDKDKAVDFSDIEALEKRLQDTQPKLLTQLFEQTSKDIAKVKIPEKLTFTIIKSMTSKIKEQMDILELVRNERIKQHGTSTEPLDMSDLDKLKGQLQTLSQYLVDDLLEDIKDSLSNIKDIATFEEESKYIEKCLDHLTGLEQTLDGSEKAIKQKEDISTYRKALIDKQEKAYPEIVQLQYKSEGLIMQLRDLCNVHHENLAEARKVRLQQLDNQNSGGGLLGGLWTVTNTLGLTTDTVHVEKMQIRMKEQVLRGFKTGLNNDKQDANQIIAFLAKKKPSELEEGLGISKENAKELHELLQQLDSKTASIDKLQENARLIDEISTKIGKEPVKQDHTITMVEEESDDIRYGL

SEQ ID NO: 4 - protein, 1495 aa, Legionella pneumophila
  SidE
MPKYVEGIELTQEGMHAIFERMGHPNITSGTIYNGEPTIDKGALDRQGFMPVLTGVSPRQDSGHWIMLIKGQGNQYFLFDPLGESSGKYYQNILAKKLPGATLSVIPNNAGLNMGLCGYWVASVGLRAHAALTQPIPPSLRNLGQTITQEMRDELTQDGSEKITQWLRAVGNEFPDGDIQPDATALRRATEKNVRIDEFQPVLTGTSPKEISINPTAPQEVSVPTWNGFSLYTDETVRNAARYAYDNYLGKPYTGTVEATPVNFGGQMVYRQHHGLAHTLRTMAYAEIIVEEARKAKLRGESLKTFADGRTLADVTPEELRKIMIAQAFFVTGRDDEESSKNYEKYHEQSRDAFLKYVEENKSTLIPDVFKDEKDVKFYADVIEDKDHKWADSPAHVLVNQGHMVDLVRVKQPPESYLEYYFSQLQPWIGSTATEAVFATQRQFFHATYEAVAGFDSENKEPHLVVDGLGRYVIGQDGNPIREESDDEDEEESGELKFFSQKKKLEENQRYMRVDEYLKLDEVQKRFPGAGKKLDGGLPGLKEYQYLQRLNSINRARCENDVDFCLGQLQTAHHQTKITPIKRAFQSSSEKARRQPNMDEIAAARIVQQIMANPDCIHDDHVFLNGQKLEEKFFRDLLAKCDMAIVGSLLNDTDIRNIDTLMQHERNTEFHSTDAKAKPVKLGETWEKTIRSGGGVTQIKHDLIFLMQNDAWYHTRVNAIAQNRDKDSTFKEVLITALMTPLTNKSLMDTSRSPAPKTLFRGLDLSEEFKNKLINQAETIIANTTEHLFTDLSTEAFKQIKLNDFSQVSARTCASTSTNIEVPRTIFGSNTIFEILDPDGLLHPKQVGTHVSGSESEYSIYLPEDVALVPIKVSFDGKTGKGKDRHIFTLVAVKSPDFTPRHESGYAVGPLLKMQTPKLEEIQRLVEQAREEPDLERVFNLQSRVARQAKFSTESGYKTFLNEKVAPVLEQSLNGLLDNNVTILGKVLSAFPSDGQWSAFNSVEARQMKIQMDAIKQMVEKKAVLEGQILPALAQCQNALEKQNIAGALQALRNIPSEKEMQTMLSISGGLRGQIQRAKQDLTETLEPLQRAITAKLVSDQEKVKVRYEKLIAGIPQQIADLEKAELADLAKVKKVVSRFNHLQEELKLLRNEKIRMHTGSEKVDFSDIAQLEAQLQKIHTKLYDAYLVELTKEISALVKEKPKNLADVKRMVSNFYAMSADIEQLRQEKIKEHGESKDPIDMSDIDKLKEELQKINQFLVKAMGTNIRVSLNQMEVKTFDAQEKEAQQNLKQLDALINKLESSDAVQKQKEELEKLNQLLVEKRKAYPAMVQLQFRSEALIIHLRELCEAHQAQMAKTRNVRAQEITNGRWKVQWLTDWVGLTTDERVTLANKEKELAKFKEDLNNDEYDLQELISNLAEKNPSELEEAIGISKESAQKLHKLLTHLNHSTTFMSKIEQRLQSIDELLNEFGKQAPRTEMIKTVEEKQGTLLRL

SEQ ID NO: 5 - protein, 76 aa, Homo sapiens [sequence omitted]

SEQ ID NO: 6 - protein, 76 aa, Mus musculus [sequence omitted]

SEQ ID NO: 7 - protein, 76 aa, Rattus norvegicus [sequence omitted]

SEQ ID NO: 8 - protein, 76 aa, Drosophila melanogaster [sequence omitted]

SEQ ID NO: 9 - protein, 76 aa, Caenorhabditis elegans
MQIFVKTLTGKTITLEVEASDTIENVKAKIQDKEGIPPDQQRLIFAGKQLEDGRTLSDYNIQKESTLHLVLRLRGG

SEQ ID NO: 10 - protein, 76 aa, Saccharomyces cerevisiae
MQIFVKTLTGKTITLEVEPSDTIDNVKSKIQDKEGIPPDQQRLIFAGKQLEDGRTLSDYNIQKESTLHLVLRLRGG

SEQ ID NO: 11 - protein, 76 aa, Artificial Sequence
  Synthetic Construct; Human UbGG/AA
MQIFVKTLTGKTITLEVEPSDTIENVKAKIQDKEGIPPDQQRLIFAGKQLEDGRTLSDYNIQKESTLHLVLRLRAA

SEQ ID NO: 12 - protein, 76 aa, Artificial Sequence; identical to SEQ ID NO: 11
  Synthetic Construct; Mouse UbGG/AA

SEQ ID NO: 13 - protein, 76 aa, Artificial Sequence; identical to SEQ ID NO: 11
  Synthetic Construct; Rat UbGG/AA

SEQ ID NO: 14 - protein, 76 aa, Artificial Sequence; identical to SEQ ID NO: 11
  Synthetic Construct; D. Melanogaster UbGG/AA

SEQ ID NO: 15 - protein, 76 aa, Artificial Sequence
  Synthetic Construct; C. elegans UbGG/AA
MQIFVKTLTGKTITLEVEASDTIENVKAKIQDKEGIPPDQQRLIFAGKQLEDGRTLSDYNIQKESTLHLVLRLRAA

SEQ ID NO: 16 - protein, 76 aa, Artificial Sequence
  Synthetic Construct; Yeast UbGG/AA
MQIFVKTLTGKTITLEVEPSDTIDNVKSKIQDKEGIPPDQQRLIFAGKQLEDGRTLSDYNIQKESTLHLVLRLRAA

SEQ ID NO: 17 - protein, 5 aa, Artificial Sequence
  Synthetic Construct; peptide linker
  REPEAT (1)..(5): Featured seqeunce can be repeated 2-5 times to form a peptide linker.
GGGGX

SEQ ID NO: 18 - protein, 15 aa, Artificial Sequence
  Synthetic Construct; peptide linker
GGGGTGGGGTGGGGT

SEQ ID NO: 19 - protein, 15 aa, Artificial Sequence
  Synthetic Construct; Biotin Motif
GLNDIFEAQKIEWHE

SEQ ID NO: 20 - protein, 15 aa, Artificial Sequence
  Synthetic Construct; Biotin Motif
ALNDIFEAQKIEWHA

SEQ ID NO: 21 - protein, 23 aa, Artificial Sequence
  Synthetic Construct; Biotin Motif
MAGGLNDIFEAQKIEWHEDTGGS

SEQ ID NO: 22 - protein, 15 aa, Artificial Sequence
  Synthetic Construct; Biotin Motif
LHHILDAQKMVWNHR

(19) United States
(12) Patent Application Publication
(10) Pub. No.: US 2022/0283179 A1
(43) Pub. Date: Sep. 8, 2022

(54) METHODS AND COMPOSITIONS FOR DETECTING PROTEIN TARGETS

(71) Applicant: THE BOARD OF REGENTS OF THE UNIVERSITY OF TEXAS SYSTEM, Austin, TX (US)

(72) Inventors: Vincent S. Tagliabracci, Dallas, TX (US); Brenden C. Park, Dallas, TX (US); Miles H. Black, Dallas, TX (US)

(21) Appl. No.: 17/684,639

(22) Filed: Mar. 2, 2022

Related U.S. Application Data
(60) Provisional application No. 63/155,580, filed on Mar. 2, 2021.

(51) Int. Cl. G01N 33/68; G01N 33/58

(52) U.S. Cl. CPC G01N 33/6845; G01N 33/6848; G01N 33/582; G01N 2458/15

(57) ABSTRACT

Compositions and methods for detection of protein interaction with a target biomolecule are provided. Compositions can include biomolecule sensors having at least a genetically modified ubiquitin peptide; an ADP-Ribosyltransferase (ART) peptide domain of a SidE-ligase protein or a homolog thereof; and a phosphodiesterase (PDE) domain of a SidE-ligase protein or a homolog thereof.

### CROSS REFERENCE TO RELATED APPLICATION

This application claims the benefit of U.S. Provisional Application No. 63/155,580 filed on Mar. 2, 2021, the disclosure of which is hereby incorporated by reference in its entirety.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 68 kilobytes in size, and titled 106546-714562_UTSD-3841-US_SequenceListing_ST25.txt.

### BACKGROUND

### 1. Field

The present inventive concept is directed to compositions and methods of using the compositions herein for the detection of protein interactions with a target biomolecule.

### 2. Discussion of Related Art

Complex protein networks are essential for sustaining the many cellular systems which play pivotal roles in life processes. Protein signaling networks are vital to cellular responses and physiological equilibriums. Dysregulation of protein signaling cascades have been associated with disease, including, for example, cancer, infectious disease, and neurodegenerative disease. Targeting proteins and their interactions is an essential strategy for the development of new therapeutics for treating a variety of diseases.

Current methods of de novo discovery of therapeutics use either a target-based screening method or a phenotypic-based screening method to identify protein-protein and/or protein-small molecule interactions to a protein target associated with a disease of interest. Phenotypic screens of small molecule compounds often produce numerous drugs that elicit clinically relevant phenotypes; however, the lack of target information causes many of the drugs that score well in phenotypic screening of disease models to fail to evolve into clinical candidates due to toxic off-target effects or high-dosage requirements. Easy target identification would allow for optimization of drug compounds to mitigate off-target effects and increase potency.

Conventional target-based approaches for identifying protein-protein and protein-small molecule interactions depend on the preservation of the interaction throughout lengthy and stringent purification steps, and frequently fail to identify transient or low-affinity interactors. In the last decade, several proximity ligation assay (PLA) techniques have emerged that catalyze covalent ligation of tags to interactors in the vicinity of a “bait” protein. However, these methods do not require direct interaction and generate reactive intermediates which may diffuse throughout the cell over the course of the experiment and result in large amounts of background labeling.

Accordingly, there is a need in the art for a simple method to aid in the identification of physiologically relevant protein-protein and protein-small molecule interactions.

### SUMMARY OF THE INVENTION

The present disclosure is based, at least in part, on the surprising discovery of a new proximity ligation assay (PLA) tool (herein referred to as “SidBait”) based on the unconventional ubiquitin ligase activity of the SidE enzymes from the pathogenic bacterium Legionella pneumophila. This tool takes advantage of the unique chemistry of SidE-catalyzed ubiquitination. In some examples herein, protein fusion constructs of ubiquitin with a SNAP-tag-small molecule conjugate bring interacting proteins in proximity of the ubiquitin molecule where the interacting proteins were first linked by SidE enzymatic activity to the bait polypeptide through a serine-phosphoribose-arginine linkage and then purified under stringent conditions. The novel PLA tools disclosed herein can facilitate final target identification by employing the uniqueness of the phosphoribose-serine bond between ubiquitin, allowing for the prey protein to be specifically cleaved and eluted from the SidBait molecule by the Legionella deubiquitinases DupA and DupB as well as with hydroxylamine (NH₂OH). Accordingly, the present disclosure provides for new PLA tools which allow for improved methods of identifying protein-protein and protein-small molecule interactions and, as such, improved de novo discovery of therapeutics.

Aspects of the present disclosure provide for biomolecule sensors as disclosed herein. In some embodiments, biomolecule sensors herein can have three constructs which together have the formula (I):

Ub-T1-L-T2-S-A;

R; and

P (I),

wherein, Ub can be a peptide having a genetically modified sequence for ubiquitin; T1 and T2 can each be an optional epitope tag; L can be a linker; S can be a self-labeling protein tag; A can be a biotin-acceptor peptide; R can be a peptide having a ADP-Ribosyltransferase (ART) domain of a SidE-ligase protein, at least one ortholog of SidE-ligase protein, or at least one paralog of a SidE-ligase protein; and P can be a peptide having a phosphodiesterase (PDE) domain of a SidE-ligase protein, at least one ortholog of SidE-ligase protein, or at least one paralog of a SidE-ligase protein. In some embodiments, Ub can be a peptide having at least 80% similarity to any one of SEQ ID NOs: 5-10. In some embodiments, Ub can have at least one genetic modification in the C-terminal peptide region. In some embodiments, R can be a peptide having a fragment of at least one paralog of a SidE effector protein from a species of Legionella. In some embodiments, R can be a peptide having a fragment of Legionella pneumophila effector SdeA. In some embodiments, R can be a peptide having an amino acid sequence of at least 30% identity to any one of SEQ ID NOs: 1-4. In some embodiments, P can be a peptide having a fragment of at least one paralog of a SidE effector protein from a species of Legionella. In some embodiments, P can be a peptide having a fragment of Legionella pneumophila effector SdeA. In some embodiments, T1 and/or T2 can be an epitope tag selected from a group of ALFA-tags, AviTags, C-tags, Calmodulin-tags, polyglutamate tags, polyarginine tags, E-tags, FLAG-tags, HA-tags, His-tags, Myc-tags, NE-tags, Rho1D4-tags, S-tags, SBP-tags, Softag 1s, Softag 3s, Spot-tags, Strep-tags, T7-tags, TC tags, Ty tags, V5 tags, VSV-tags, and Xpress tags. In some embodiments, L can be a peptide having 2 to 20 amino acids. In some embodiments, L can be a peptide having an amino acid sequence of [GGGX]ₙ, wherein X is A, T or S; and wherein n is 2-5. In some embodiments, L can be a peptide having an amino acid sequence of SEQ ID NO: 18. In some embodiments, S can be a self-labeling protein tag selected from a group of Halo-tags, SNAP-tags, CLIP-tags, ACP-tags, MCP-tags, and UV-cross-linking capable tags. In some embodiments, UV-cross-linking capable tags can include one or more of any combination of aryl azides, fluorinated aryl azides, azido-methyl-coumarins, benzophenones, anthraquinones, diazo compounds, diazirines, psoralens, 5-halo-uridines, 5-halo-cytosines, 7-halo-adenosines, 2-nitro-5-azidobenzoyls, fluorinated aryl azides, amino-benzophenones, and derivatives thereof. In some embodiments, A can be a biotin-acceptor peptide having 10 to 400 amino acids. In some embodiments, A can be a biotin-acceptor peptide having an amino acid sequence of SEQ ID NO: 19.

In some embodiments, any one or more of the components of formula (I) disclosed herein may be generated separately. In some aspects, any one or more of the components of formula (I) disclosed herein may be generated separately and further ligated together. In some examples, one or more components of formula (I) may be first generated and then ligated together at any point in time after generating the one or more components. In some other examples, two or more components of formula (I) may be generated in complex together and then ligated to one or more components generated separately. In still some other examples, Ub-T1-L-T2-S-A, R and/or P of formula (I) may be generated separately. In some examples, Ub-T1-L-T2-S-A, R and/or P of formula (I) may be generated separately and at least two of the components ligated together at any point in time after generating the one or more components. In some other examples, Ub-T1-L-T2-S-A, R and P of formula (I) may be generated separately and ligated together at any point in time after generating the components.

Aspects of the present disclosure provide systems as disclosed herein for proximity-based labeling of biomolecules. In some embodiments, systems herein can have (a) at least one construct having a genetically modified ubiquitin (Ub), a self-labeling protein tag, a linker, or a combination thereof; (b) a biomolecule derivative; and (c) at least two protein domains of a SidE-ligase protein, an ortholog of SidE-ligase protein, or a paralog of a SidE-ligase protein. In some embodiments, systems herein can further have at least one an epitope tag. In some embodiments, systems herein can further have at least one biotin-acceptor peptide.

Aspects of the present disclosure provide methods for detecting a protein interaction with a target biomolecule as disclosed herein. In various embodiments, methods herein can have one or more steps of the following: (a) contacting a sample having a target biomolecule with a biomolecule sensor, the biomolecule sensor comprising three constructs, the three constructs which together have the formula (I):

Ub-T1-L-T2-S-A;

R; and

P (I)

wherein, Ub can be a peptide having a genetically modified sequence for ubiquitin; T1 and T2 can each be an optional epitope tag; L is a linker; S can be a self-labeling protein tag or a target protein; A can be biotin-acceptor peptide; R can be a peptide having a ADP-Ribosyltransferase (ART) domain of a SidE-ligase protein, at least one ortholog of SidE-ligase protein, or at least one paralog of a SidE-ligase protein; and P can be a peptide having a phosphodiesterase (PDE) domain of a SidE-ligase protein, at least one ortholog of SidE-ligase protein, or at least one paralog of a SidE-ligase protein; and (b) detecting whether the target biomolecule is bound to 5, indicating that the protein interaction with a target biomolecule can be present in the sample. In some embodiments, a target biomolecule for use herein can be derivative of the target biomolecule. In some examples, a derivative of the target biomolecule can be a benzylguanine (BG) derivative, a chloropyrimidine (CLP) derivative, or a combination thereof. In some examples, a derivative of the target biomolecule can have at least one cross-linking side group. In some embodiments, a target biomolecule herein can be bound to S by a phosphoribose linkage. In some embodiments, Ub can be ADP-ribosylated by R.

Aspects of the present disclosure provide kits having compositions disclosed herein and for use in methods disclosed herein.

### BRIEF DESCRIPTION OF THE DRAWINGS

Embodiments of the present inventive concept are illustrated by way of example in which like reference numerals indicate similar elements and in which:

FIGS. 1A-1C depict images illustrating the SidE family of Legionella effectors as all-in-one Ub ligases in accordance with certain aspects of the present disclosure. FIG. 1A shows a schematic representation of the domain architecture of the SidE-family Ub ligases having an N-terminal DUB domain, a C-terminal CTD, and domains responsible for ubiquitination including a PDE domain and an ART domain. FIGS. 1B and 1C show a schematic illustrating a reaction catalyzed by the SidE effectors in general (FIG. 1B) and a closer view of the chemistry of the reaction (FIG. 1C). In the first step of the reaction, the ART domain in SidE uses NAD+ as a cofactor to transfer ADP-ribose to Arg42 in Ub. The PDE domain in SidE then hydrolyzes the phosphodiester bond to generate phosphoribosylated (pR) Ub, or links the Ub to target proteins via a Ser-pR-Ub linkage. (NAD; nicotinamide adenine dinucleotide).

FIGS. 2A and 2B depict images illustrating the SidBait approach to discover protein targets of small molecule drugs in accordance with certain aspects of the present disclosure. FIG. 2A shows an overall schematic of the SidBait technology. In the first reaction, (1) a Ub^{GG/AA}-SNAP fusion protein is ADP ribosylated by the SidE ART domain. Next, (2) a chloropyrimidine (CLP) derivative of a small molecule is conjugated to the SNAP tag, which (3) is then incubated with a cell lysate and the small molecule engages its target. Next, (4) the SidE PDE domain is then added to covalently link the drug target to the bait. Finally, (5) the drug target is affinity purified by avidin pulldown and subjected to LC/MS/MS. FIG. 2B shows a schematic representation of Ub^{GG/AA}-SNAP depicting the N-terminal 6X His tag, the Ub^{GG/AA}, the HA tag, the flexible linker (GGGGT)×3, the Flag tag, the SNAP tag, and the C-terminal Avi tag.

FIGS. 3A-3C depict images illustrating the characterization of SidBait constructs in accordance with certain aspects of the present disclosure. FIG. 3A shows an intact mass LC/MS spectra of Ub^{GG/AA}-SNAP and Ub^{GG/AA}-SNAP treated with SdeA^{ART} and E. coli BirA to generate SidBait. Note the two spectra were overlaid on the same graph and depict the increase in mass of ˜767.61 Da, which corresponded to the addition of an ADP-ribose (˜541 Da) and a biotin (˜226 Da). FIG. 3B shows an image of a representative Coomassie-stained SDS-PAGE gel (upper panel) of SidBait and SidBait^{C145A} following incubation with SNAP Cell Oregon Green dye. FIG. 3B also shows an image of a representative the SDS-PAGE gel analyzed for Oregon Green fluorescence (lower panel) to detect conjugation of the dye to the SNAP tag in SidBait. FIG. 3C shows an image of a representative Coomassie-stained SDS-PAGE gel depicting the products of the SidBait crosslinking reaction. Ub^{GG/AA} and SidBait (and the C145A mutants) were incubated with SdeA^{PDE} and the reaction products were separated by SDS PAGE and visualized by Coomassie staining. Ub and ADP ribosylated Ub (ADPR-Ub) were included as controls. Schematic representation of the reaction components are also shown in FIG. 3C.

FIGS. 4A-4F depict images illustrating the use of the SidBait constructs, SidBait-MLN8237 and SidBait-BI2536, to detect protein targets in accordance with certain aspects of the present disclosure. FIG. 4A shows an image of the chemical structures of CLP-MLN8237. FIG. 4B shows an image of the chemical structure of CLP-BI2536. FIGS. 4C and 4D show images of representative immunoblots of HEK293A lysates incubated with SidBait-MLN8237 (FIG. 4C) and SidBait-BI2536 (FIG. 4D) before (−) and after (+) treatment with SdeA^{PDE} where the blots were probed for AurA, PLK1, flag, or GAPDH where indicated. GAPDH is shown as a loading control. Lysates were separated by SDS-PAGE and proteins were detected by immunoblotting using the indicated antibodies. FIGS. 4E and 4F depict graphs showing MS enrichment plots of avidin pulldowns following SdeA^{PDE} treatment of HEK293A lysates incubated with WT/C145A SidBait-MLN8237 (FIG. 4E) or WT/C145A SidBait-BI2536 (FIG. 4F) where individual proteins are plotted as dots and the known targets of the drugs are highlighted in red.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized, and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, at least in part, on the surprising discovery of a new proximity ligation assay (PLA) tool (herein referred to as “SidBait”) based on the unconventional ubiquitin ligase activity of SidE enzymes from the pathogenic bacterium Legionella pneumophila. The present disclosure provides for biomolecule sensor compositions, systems for proximity-based labeling of biomolecules, methods of making biomolecule sensor compositions disclosed herein, and methods of using biomolecule sensor compositions disclosed herein.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mM includes all values from 1 mM to 9 mM, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The term “biomolecule” as used herein refers to, but is not limited to, proteins, enzymes, antibodies, DNA, siRNA, and small molecules. “Small molecules” as used herein can refer to chemicals, compounds, drugs, and the like.

The term “modify” or “modifying” and grammatical variations thereof, when used in reference to any of the compositions (e.g., proteins, protein domains, peptides, peptide fragments, polypeptide sequences) disclosed herein means that the modified composition deviates from a reference composition.

The term “recombinant” as used herein refers to an introduction of a heterologous nucleic acid or amino acid, and/or the alteration of a native nucleic acid or protein.

The term “solid support” as used herein refers to a material having a rigid or semi-rigid surface. Such materials will preferably take the form of small beads, pellets, disks, chips, or wafers, although other forms may be used.

The term “surface” as used herein refers to any generally two-dimensional structure on a solid substrate and may have steps, ridges, kinks, terraces, and the like without ceasing to be a surface.

### II. Compositions and Systems

The present disclosure provides for biomolecule sensor compositions and systems for proximity-based labeling of a biomolecule.

### (a) SidE-Family of Ubiquitin (Ub) Ligases

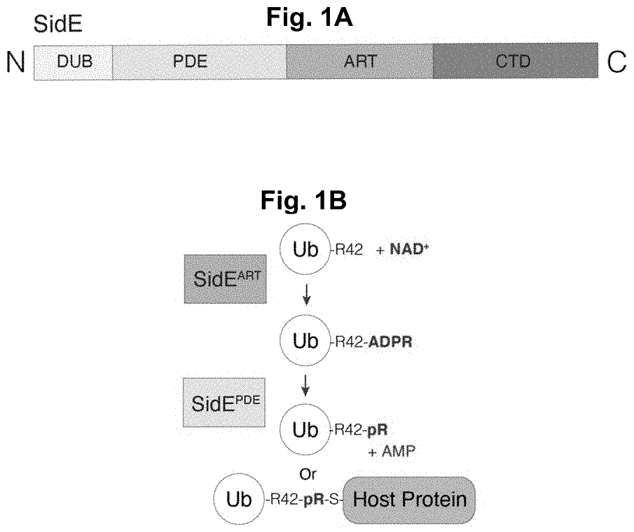

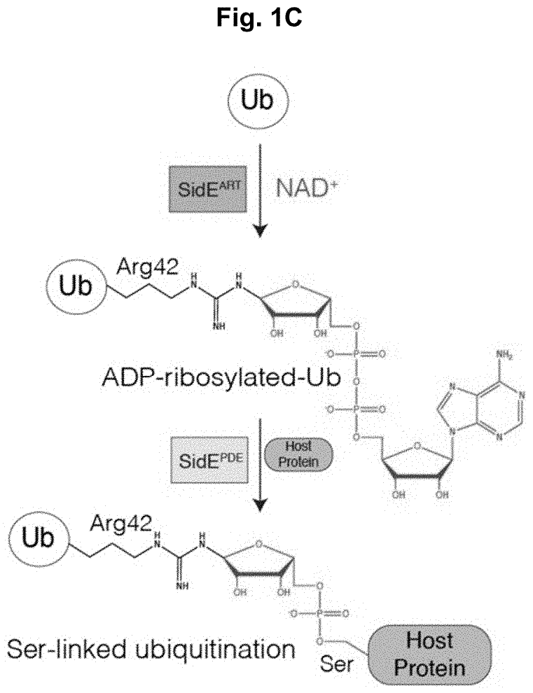

In various embodiments, compositions herein can have at least one protein, protein domain, peptide, peptide fragment, polypeptide sequence, or a combination thereof corresponding to a protein belonging to a SidE-family of ubiquitin (Ub) ligases. The SidE-family of ubiquitin (Ub) ligases are type 4 secretion system effector proteins found in the Gram-negative human bacterial pathogen Legionella pneumophila. The SidE-family consists of four functionally redundant proteins (SdeA, SdeB, SdeC and SidE) that contain an N-terminal deubiquitinase domain an HD-like phosphodiesterase (PDE) domain, an ADP Ribosyltransferase (ART) domain and a C-terminal domain (CTD) containing a coiled coil region (See e.g., FIG. 1A).

In some examples, a protein, protein domain, peptide, peptide fragment, polypeptide sequence, or a combination thereof suitable for compositions herein can be one or more homologs belonging to a SidE-family of ubiquitin (Ub) ligases. As used herein, the term “homologs” refers to a gene related to a second gene by descent from a common ancestral DNA sequence. In some examples, a protein, protein domain, peptide, peptide fragment, polypeptide sequence, or a combination thereof suitable for compositions herein can be one or more orthologs belonging to a SidE-family of ubiquitin (Ub) ligases. In some examples, a protein, protein domain, peptide, peptide fragment, polypeptide sequence, or a combination thereof suitable for compositions herein can be one or more paralogs belonging to a SidE-family of ubiquitin (Ub) ligases. As used herein, “orthologs” refers to homologs in different species that catalyze the same reaction, and “paralogs” refer to homologs in the same species that may or may not catalyze the same reaction.

In some embodiments, a protein, protein domain, peptide, peptide fragment, polypeptide sequence, or a combination thereof suitable for compositions herein can be from a PDE domain, an ART domain, a CTD domain, or a combination thereof of one or more homologs of a SidE-family of ubiquitin (Ub) ligases. In some embodiments, a protein, protein domain, peptide, peptide fragment, polypeptide sequence, or a combination thereof suitable for compositions herein can be from a PDE domain, an ART domain, a CTD domain, or a combination thereof of one or more orthologs of a SidE-family of ubiquitin (Ub) ligases. In some embodiments, a protein, protein domain, peptide, peptide fragment, polypeptide sequence, or a combination thereof suitable for compositions herein can be from a PDE domain, an ART domain, a CTD domain, or a combination thereof of one or more paralogs of a SidE-family of ubiquitin (Ub) ligases.

In some examples, a protein, protein domain, peptide, peptide fragment, polypeptide sequence, or a combination thereof suitable for compositions herein can be one or more paralogs belonging to a SidE-family of ubiquitin (Ub) ligases from a species of Legionella. In some examples, a protein, protein domain, peptide, peptide fragment, polypeptide sequence, or a combination thereof suitable for compositions herein can be a PDE domain, an ART domain, a CTD domain, or a combination thereof of one or more paralogs belonging to a SidE-family of ubiquitin (Ub) ligases from a species of Legionella.

In some examples, a protein, protein domain, peptide, peptide fragment, polypeptide sequence, or a combination thereof suitable for compositions herein can belong to a SidE-family of ubiquitin (Ub) ligases from Legionella pneumophila. In some examples, a protein, protein domain, peptide, peptide fragment, polypeptide sequence, or a combination thereof suitable for compositions herein can be a PDE domain, an ART domain, a CTD domain, or a combination thereof of one or more paralogs belonging to a SidE-family of ubiquitin (Ub) ligases from Legionella pneumophila.

In some embodiments, a protein, protein domain, peptide, peptide fragment, polypeptide sequence, or a combination thereof suitable for compositions herein can be from a SdeA protein, a SdeB protein, a SdeC protein, a SidE protein, or a combination thereof. In some examples, a protein, protein domain, peptide, peptide fragment, polypeptide sequence, or a combination thereof suitable for compositions herein can be from a homolog of SdeA protein, a SdeB protein, a SdeC protein, a SidE protein, or a combination thereof. In some examples, a protein, protein domain, peptide, peptide fragment, polypeptide sequence, or a combination thereof suitable for compositions herein can be from an ortholog of SdeA protein, a SdeB protein, a SdeC protein, a SidE protein, or a combination thereof. In some examples, a protein, protein domain, peptide, peptide fragment, polypeptide sequence, or a combination thereof suitable for compositions herein can be from a paralog of SdeA protein, a SdeB protein, a SdeC protein, a SidE protein, or a combination thereof.

In some embodiments, a protein, protein domain, peptide, peptide fragment, polypeptide sequence, or a combination thereof suitable for compositions herein can have an identical sequence similarity to a SdeA protein (SEQ ID NO. 1). In some examples, a SdeA protein for use herein can be genetically modified. In some examples, a SdeA protein for use herein can be variant of SdeA. In some embodiments, a protein, protein domain, peptide, peptide fragment, polypeptide sequence, or a combination thereof suitable for compositions herein can have at least about 30% (e.g., about 30%, 40%, 50%, 60%, 70%, 80%, 90%, 100%) sequence similarity to a SdeA protein (SEQ ID NO. 1). In some embodiments, a protein, protein domain, peptide, peptide fragment, polypeptide sequence, or a combination thereof suitable for compositions herein can have about 50% to about 99% sequence similarity to a SdeA protein (SEQ ID NO. 1). In some examples, at least one effector domain of a SdeA protein can be used in compositions disclosed herein. In some examples, a protein, protein domain, peptide, peptide fragment, polypeptide sequence, or a combination thereof suitable for compositions herein can be a PDE domain, an ART domain, and/or a CTD domain of SdeA.

In some embodiments, a protein, protein domain, peptide, peptide fragment, polypeptide sequence, or a combination thereof suitable for compositions herein can have an identical sequence similarity to a SdeB protein (SEQ ID NO. 2). In some examples, a SdeB protein for use herein can be genetically modified. In some examples, a SdeB protein for use herein can be variant of SdeB. In some embodiments, a protein, protein domain, peptide, peptide fragment, polypeptide sequence, or a combination thereof suitable for compositions herein can have at least about 30% (e.g., about 30%, 40%, 50%, 60%, 70%, 80%, 90%, 100%) sequence similarity to a SdeB protein (SEQ ID NO. 2). In some embodiments, a protein, protein domain, peptide, peptide fragment, polypeptide sequence, or a combination thereof suitable for compositions herein can have about 50% to about 99% sequence similarity to a SdeB protein (SEQ ID NO. 2). In some examples, at least one effector domain of a SdeB protein can be used in compositions disclosed herein. In some examples, a protein, protein domain, peptide, peptide fragment, polypeptide sequence, or a combination thereof suitable for compositions herein can be a PDE domain, an ART domain, and/or a CTD domain of SdeB.

In some embodiments, a protein, protein domain, peptide, peptide fragment, polypeptide sequence, or a combination thereof suitable for compositions herein can have an identical sequence similarity to a SdeC protein (SEQ ID NO. 3). In some examples, a SdeC protein for use herein can be genetically modified. In some examples, a SdeC protein for use herein can be variant of SdeC. In some embodiments, a protein, protein domain, peptide, peptide fragment, polypeptide sequence, or a combination thereof suitable for compositions herein can have at least about 30% (e.g., about 30%, 40%, 50%, 60%, 70%, 80%, 90%, 100%) sequence similarity to a SdeC protein (SEQ ID NO. 3). In some embodiments, a protein, protein domain, peptide, peptide fragment, polypeptide sequence, or a combination thereof suitable for compositions herein can have about 50% to about 99% sequence similarity to a SdeC protein (SEQ ID NO. 3). In some examples, at least one effector domain of a SdeC protein can be used in compositions disclosed herein. In some examples, a protein, protein domain, peptide, peptide fragment, polypeptide sequence, or a combination thereof suitable for compositions herein can be a PDE domain, an ART domain, and/or a CTD domain of SdeC.

In some embodiments, a protein, protein domain, peptide, peptide fragment, polypeptide sequence, or a combination thereof suitable for compositions herein can have an identical sequence similarity to a SidE protein (SEQ ID NO, 4). In some examples, a SidE protein for use herein can be genetically modified. In some examples, a SidE protein for use herein can be variant of SidE. In some embodiments, a protein, protein domain, peptide, peptide fragment, polypeptide sequence, or a combination thereof suitable for compositions herein can have at least about 30% (e.g., about 30%, 40%, 50%, 60%, 70%, 80%, 90%, 100%) sequence similarity to a SidE protein (SEQ ID NO. 4). In some embodiments, a protein, protein domain, peptide, peptide fragment, polypeptide sequence, or a combination thereof suitable for compositions herein can have about 50% to about 99% sequence similarity to a SidE protein (SEQ ID NO. 4). In some examples, at least one effector domain of a SidE protein can be used in compositions disclosed herein. In some examples, a protein, protein domain, peptide, peptide fragment, polypeptide sequence, or a combination thereof suitable for compositions herein can be a PDE domain, an ART domain, and/or a CTD domain of SidE.

In some embodiments, a protein, protein domain, peptide, peptide fragment, polypeptide sequence, or a combination thereof suitable for compositions herein can have a truncation of a SdeA protein, a SdeB protein, a SdeC protein, a SidE protein, or any combination thereof. In some examples, a truncation herein can have up to half of a N-terminal amino acid sequence of a SdeA protein, a SdeB protein, a SdeC protein, or a SidE protein. In some examples, a truncation herein can have between about 10% to about 95% of a N-terminal amino acid sequence of a SdeA protein, a SdeB protein, a SdeC protein, or a SidE protein. In some examples, a truncation herein can have up to half of a C-terminal amino acid sequence of a SdeA protein, a SdeB protein, a SdeC protein, or a SidE protein. In some examples, a truncation herein can have between about 10% to about 95% of a C-terminal amino acid sequence of a SdeA protein, a SdeB protein, a SdeC protein, or a SidE protein. In some embodiments, a protein, protein domain, peptide, peptide fragment, polypeptide sequence, or a combination thereof suitable for compositions herein can have at least two truncations. In some embodiments, at least two truncations can be fused together to form a protein, protein domain, peptide, peptide fragment, polypeptide sequence, or a combination thereof suitable for compositions herein. In some examples, at least two truncations can be fused together to form a protein, protein domain, peptide, peptide fragment, polypeptide sequence, or a combination thereof suitable for compositions herein wherein at least one of the truncations has between about 10% to about 95% of a N-terminal amino acid sequence of a SdeA protein, a SdeB protein, a SdeC protein, or a SidE protein and at least one of the other truncations has between about 10% to about 95% of a C-terminal amino acid sequence of a SdeA protein, a SdeB protein, a SdeC protein, or a SidE protein. In some examples, at least two truncations can be fused together to form a protein, protein domain, peptide, peptide fragment, polypeptide sequence, or a combination thereof suitable for compositions herein wherein at least one of the truncations has up to half of a N-terminal amino acid sequence of a SdeA protein, a SdeB protein, a SdeC protein, or a SidE protein and at least one of the other truncations has up to half of a C-terminal amino acid sequence of a SdeA protein, a SdeB protein, a SdeC protein, or a SidE protein.

In certain embodiments, any of the proteins, protein domains, peptides, peptide fragments, and polypeptide sequences disclosed herein can be prepared by recombinant technology as exemplified below.

### (b) Ubiquitin

In various embodiments, compositions herein can have at least one peptide having an amino acid sequence for ubiquitin. Ubiquitin is a protein that exists in all eukaryotic cells. Ubiquitin can perform a multitude of functions through conjugation to a large range of target proteins. Native ubiquitin protein can have about 74-77 amino acids and have a molecular mass of about 8 to about 9 kDa. The amino acid sequence of ubiquitin is highly conserved across different organisms. As such, some embodiments can have compositions herein having at least one peptide having an amino acid sequence for ubiquitin from a variety of organisms including, but not limited to, H. sapiens, M. musculus, R. norvegicus, D. melanogaster, C. elegans, and S. cerevisiae. Table 1 provides, in part, the amino acid sequences for ubiquitin of the indicated organisms.

TABLE 1

| Organism/ | | SEQ ID |
|---|---|---|
| Construct | Sequence | NO. |
| H. sapiens | MQIFVKTLTGKTITLEVEPSDTIENVKAKIQDKEGIPP | 5 |
| | DQQRLIFAGKQLEDGRTLSDYNIQKESTLHLVLRLRGG | |
| M. musculus | MQIFVKTLTGKTITLEVEPSDTIENVKAKIQDKEGIPP | 6 |
| | DQQRLIFAGKQLEDGRTLSDYNIQKESTLHLVLRLRGG | |
| R. nolvegicus | MQIFVKTLTGKTITLEVEPSDTIENVKAKIQDKEGIPP | 7 |
| | DQQRLIFAGKQLEDGRTLSDYNIQKESTLHLVLRLRGG | |
| D. Melanogaster | MQIFVKTLTGKTITLEVEPSDTIENVKAKIQDKEGIPP | 8 |
| | DQQRLIFAGKQLEDGRTLSDYNIQKESTLHLVLRLRGG | |
| C. elegans | MQIFVKTLTGKTITLEVEASDTIENVKAKIQDKEGIPP | 9 |
| | DQQRLIFAGKQLEDGRTLSDYNIQKESTLHLVLRLRGG | |
| S. cerevisiae | MQIFVKTLTGKTITLEVEPSDTIDNVKSKIQDKEGIPP | 10 |
| | DQQRLIFAGKQLEDGRTLSDYNIQKESTLHLVLRLRGG | |
| Human Ub^{GG/AA} | MQIFVKTLTGKTITLEVEPSDTIENVKAKIQDKEGIPP | 11 |
| | DQQRLIFAGKQLEDGRTLSDYNIQKESTLHLVLRLRAA | |
| Mouse Ub^{GG/AA} | MQIFVKTLTGKTITLEVEPSDTIENVKAKIQDKEGIPP | 12 |
| | DQQRLIFAGKQLEDGRTLSDYNIQKESTLHLVLRLRAA | |
| Rat Ub^{GG/AA} | MQIFVKILTGKTITLEVEPSDTIENVKAKIQDKEGIPP | 13 |
| | DQQRLIFAGKQLEDGRTLSDYNIQKESTLHLVLRLRAA | |
| D. Melanogaster Ub^{GG/AA} | MQIFVKTLTGKTITLEVEPSDTIENVKAKIQDKEGIPP | 14 |
| | DQQRLIFAGKQLEDGRTLSDYNIQKESTLHLVLRLRAA | |
| C. elegans Ub^{GG/AA} | MQIFVKTLTGKTITLEVEASDTIENVKAKIQDKEGIPP | 15 |
| | DQQRLIFAGKQLEDGRTLSDYNIQKESTLHLVLRLRAA | |
| Yeast Ub^{GG/AA} | MQIFVKTLTGKTITLEVEPSDTIDNVKSKIQDKEGIPP | 16 |
| | DQQRLIFAGKQLEDGRTLSDYNIQKESTLHLVLRLRAA | |

In some embodiments, compositions herein can have at least one Ub peptide having any one of SEQ ID NO: 5-10. In some embodiments, compositions herein can have at least one Ub peptide that has been genetically modified. In some examples, at least one amino acid in the C-terminus of an Ub peptide used herein can be genetically modified. In some examples, at least one amino acid in the N-terminus of an Ub peptide used herein can be genetically modified.

In some embodiments, compositions herein can have at least one genetically modified Ub peptide having at least about 40% (e.g., about 40%, 50%, 60%, 70%, 80%, 90%, 95%) similarity to any one of SEQ ID NOs: 5-10. In some examples, compositions herein can have at least one genetically modified Ub peptide having at least about 80% similarity to any one of SEQ ID NOs: 5-10. In some examples, compositions herein can have at least one genetically modified Ub peptide having about 80% to about 99% similarity to any one of SEQ ID NOs: 5-10.

In some embodiments, compositions herein can have at least one genetically modified Ub peptide wherein about 1 to about 5 amino acids of the C-terminal end are mutated. In some examples, compositions herein can have at least one genetically modified Ub peptide wherein two C-terminal end amino acids residues are mutated. In some examples, compositions herein can have at least one genetically modified Ub peptide wherein the C-terminal diglycine can be mutated to dialanine (Ub^{GG/AA}). In some exemplary examples, compositions herein can have at least one genetically modified Ub peptide having any one of SEQ ID NOs: 11-16, as provided in Table 1.

In some embodiments, compositions herein can have at least one genetically modified Ub peptide wherein about 1 to about 50 amino acids (e.g., about 1, about 5, about 10, about 15, about 20, about 25, about 30, about 35, about 40, about 45, about 50) are added to the C-terminal end of an Ub peptide. In some examples, compositions herein can have at least one genetically modified Ub peptide wherein about 1 to about 50 amino acids are added to the C-terminal end of an Ub peptide, wherein the Ub peptide can have any one of SEQ ID NOs: 5-16 as provided in Table 1. In some examples, compositions herein can have at least one genetically modified Ub peptide with amino acids having a sequence of GGAA(X)ₙ added to the C-terminal end of the Ub peptide, wherein “n” can be an amino acid sequence having up to about 1 to about 50 amino acid residues. In some examples, compositions herein can have at least one genetically modified Ub peptide with amino acids having a sequence of GGAA(X)ₙ added to the C-terminal end of the Ub peptide, wherein “n” is an amino acid sequence having about 15 amino acid residues.

### (c) Tags

In various embodiments, compositions herein can have at least one tag. As used herein, the term “tag” refers to a detectable moiety that can be atoms, molecules, peptides, or a collection of atoms, molecules, and peptides. A tag used herein can provide an optical, electrochemical, magnetic, or electrostatic (e.g., inductive, capacitive) signature which can be detected by methods known in the art. A tag can also be used as a means to enrich, to purify, or a combination thereof of its tagged biomolecule. Methods of attaching a tag to compositions herein are known in the art and can include, but are not limited to, recombinant expression, cross-linking labeling methods, chemical labeling methods, enzyme-catalyzed labeling methods, and the like. In some examples, a tag used herein can be permanently fixed to its tagged biomolecule. In some examples, a tag used herein can be removed from its tagged biomolecule. In some examples, a tag used herein can be attached in a cis position. In some examples, a tag used herein can be attached in a trans position. Methods of removing a tag from a composition herein are known in the art and can include, but are not limited to, chemical cleavage, enzymatic cleavage, and the like.

In some embodiments, the compositions herein can have at least three tags. In some embodiments, the at least three tags can be the same type of tag. In some embodiments the at least three tags can be different types of tags. In some embodiments the at least three tags can have two of the same type of flag whereas a third flag is of a different type. In some embodiments, the compositions herein can have at least two tags. In some embodiments, the at least two tags can be the same type of tag. In some embodiments the at least two tags can be different types of tags. In some embodiments, the compositions herein can have one tag. In some embodiments, the compositions herein may not require a tag. In some examples, the compositions herein can have a self-labeling protein tag and optionally have one or two epitope tags. In some examples, the compositions herein can optionally have one or two epitope tags without having a self-labeling protein tag.

In certain embodiments, a tag used herein can be a peptide tag. In some examples, a peptide tag used herein can have anywhere from about 2 amino acid residues to about 500 amino acid residues.

In some embodiments, a peptide tag for use herein can be an epitope tag. As used herein, an “epitope tag” refers to a tag that allows for detection and/or purification of its attached composition by immunoassay and/or fluorescence. Non-limiting examples of epitope tags suitable for use herein can include, but are not limited to, ALFA-tags, AviTags, C-tags, Calmodulin-tags, polyglutamate tags, polyarginine tags, E-tags, FLAG-tags, HA-tags, His-tags, Myc-tags, NE-tags, Rho1D4-tags, S-tags, SBP-tags, Softag 1s, Softag 3s, Spot-tags, Strep-tags, T7-tags, TC tags, Ty tags, V5 tags, VSV-tags, and Xpress tags. The skilled artisan would readily recognize other useful epitope tags that are not mentioned above, which may be employed in the operation of the present disclosure. In some examples, an epitope tag can be modified to make it more suitable for use in the compositions and methods disclosed herein.

In some embodiments, compositions herein can include about 1 to about 10 epitope tags. In some examples, compositions herein can include about 1 to about 2 epitope tags. In some examples, compositions herein do not have an epitope tag. In some examples, an epitope tag can be inserted at any amino acid residue of a protein, protein domain, peptide, peptide fragment, or polypeptide sequence disclosed herein. In some examples, an epitope tag can be inserted at a C terminal end, at a N terminal end, or at both the C terminal and N terminal ends of a protein, protein domain, peptide, peptide fragment, or polypeptide sequence disclosed herein. In some examples, an epitope tag can be inserted at a C terminal end, at a N terminal end, at both the C terminal and N terminal ends, and/or at any amino acid residue of any of the peptides having an amino acid sequence for ubiquitin disclosed herein.

In certain embodiments, a tag used herein can be a self-labeling protein tag. As used herein, a “self-labeling protein tag” can refer to a tag that labels itself in the presence of its ligand, typically a low molecular weight compound, in a covalent manner. Non-limiting examples self-labeling protein tags suitable for use herein can include Halotags, SNAP-tags, CLIP-tags, ACP-tags and MCP-tags. In some embodiments, self-labeling protein tags can be UV-cross-linking capable tags. In some embodiments, UV-cross-linking capable tags can be one or more photochemical reactive groups. In some examples, photochemical reactive groups suitable for use herein as UV-cross-linking capable tags can include aryl azides, fluorinated aryl azides, azido-methyl-coumarins, benzophenones, anthraquinones, diazo compounds, diazirines, psoralens, 5-halo-uridines, 5-halo-cytosines, 7-halo-adenosines, 2-nitro-5-azidobenzoyls, fluorinated aryl azides, amino-benzophenones, or derivatives thereof. Non-limiting examples of photochemical reactive groups suitable for use herein as UV-cross-linking capable tags can include ABH (p-Azidobenzoyl Hydrazide); ANB-NOS (N-5-Azido-2-nitrobenzyloxysuccinimide); APG (p-Azidophenyl Glyoxal Monohydrate); APDP (N-(4-[p-Azidosalicylamido]butyl)-3′-(2′-pyridyldithio) Propionamide); BASED (bis([beta]-[4-Azidosalicylamido]-ethyl) Disulfide); NHS-ASA (N-Hydroxysuccinimideyl-4-azidosalicyclic Acid); Sulfo HSAB (N-Hydroxysulfosuccinimidyl-4-azidosalicylic Acid); SDA [(NHS-Diazirine), (succinimidyl 4,4′-azipentanoate)]; Sulfo SAND (Sulfosuccinimidyl 2-(m-Azido-o-nitrobenzamido)-ethyl-1,3′-propionate); Sulfo SANPAH (Sulfosuccinimidyl 6-(4′-Azido-2′-nitrophenylamino) Hexanoate); Sulfo SADP (Sulfosuccinimidyl (4-Azidophenyl dithio)propionate); Sulfo SASD (Sulfosuccinimidyl-2-(p-Azidosalicylamido)ethyl-1,3-Dithiopropionate); SDAD [(NHS-SS-Diazirine), (succinimidyl 2-((4,4′-azipentanamido)ethyl)-1,3′-dithiopropionate)]; Sulfo-LC-SDA [(Sulfo-NHS-LC-Diazirine), (sulfosuccinimidyl 6-(4,4′-azipentanamido)hexanoate)]; Sulfo-SDAD [(Sulfo-NHS-SS-Diazirine), (sulfosuccinimidyl 2-((4,4′-azipentanamido)ethyl)-1,3′-dithiopropionate)]; Sulfo-SDA [(Sulfo-NHS-Diazirine), (sulfosuccinimidyl 4,4′-azipentanoate)]; LC-SDA [(NHS-LC-Diazirine), (succinimidyl 6-(4,4′-azipentanamido)hexanoate)], SPB (succinimidyl-[4-(psoralen-8-yloxy)]butyrate); 4-(4-(Prop-2-yn-1-yloxy)benzoyl)benzoic acid; 3-(3-Methyl-3H-diazirin-3-yl)-N-(3-(triethoxysilyl)propyl)propanamide; 4-(N-Maleimido)benzophenone; 3-(Fluorosulfonyl)-5-((trimethylsilyl)ethynyl)benzoic acid; 4-Benzoylbenzoic acid N-succinimidyl ester; 5-Azido-2-nitrobenzoic acid N-hydroxysuccinimide ester; and the like. The skilled artisan would readily recognize other useful self-labeling protein tags that are not mentioned above, which may be employed in the operation of the present disclosure.

In some embodiments, a self-labeling protein tag for use herein can be genetically fused to any of the proteins, protein domains, peptides, peptide fragments, or polypeptide sequences disclosed herein. In some embodiments, a self-labeling protein tag for use herein can be cross-linked to any of the proteins, protein domains, peptides, peptide fragments, or polypeptide sequences disclosed herein. In some examples, a self-labeling protein tag can be cross-linked to any of the proteins, protein domains, peptides, peptide fragments, or polypeptide sequences disclosed herein by photo-crosslinking methods, UV cross-linking methods, chemical cross-linking methods; enzymatic cross-linking methods; and the like.

In some embodiments, a self-labeling protein tag suitable for use herein can be genetically modified at least about one to about 5 amino acid residues. The skilled artisan would readily recognize genetic modification of self-labeling protein tags herein can be undertaken to optimize the tags' performance in the methods disclosed herein and/or for ease of forming compositions disclosed herein.

In some embodiments, compositions herein can include about 1 to about 10 self-labeling protein tags. In some examples, compositions herein can include about 1 to about 2 self-labeling protein tags. In some examples, compositions herein do not have a self-labeling protein tag. In some examples, a self-labeling protein tag can be inserted at any amino acid residue of a protein, protein domain, peptide, peptide fragment, or polypeptide sequence disclosed herein. In some examples, a self-labeling protein tag can be inserted at a C terminal end, at a N terminal end, or at both the C terminal and N terminal ends of a protein, protein domain, peptide, peptide fragment, or polypeptide sequence disclosed herein. In some examples, a self-labeling protein tag can be inserted at a C terminal end, at a N terminal end, at both the C terminal and N terminal ends, and or at any amino acid residue of any of the peptides herein having an amino acid sequence for ubiquitin.

### (d) Linkers

In various embodiments, compositions herein can have at least one linker. In some embodiments, a linker suitable for use herein can be a chemical linker, a polymer-base linker, a peptide linker, or a combination thereof. In some embodiments, compositions herein can have at least one peptide linker. In some embodiments, a peptide linker for use herein can have about 20 or fewer amino acids. One of skill in the art can appreciate that the length of the linker can be optimized depending on the desired use of the linker (e.g., desired flexibility of the linker, desired interactions of the linker and the linked biomolecule, and the like.)

In some embodiments, a peptide linker for use herein can have at least two amino acids in length. In some embodiments, a peptide linker for use herein can have between 2 and 20 amino acids, wherein at least 50% of said amino acids are G residues. In some embodiments, a peptide linker for use herein can have the amino acid sequence [GGGGX]n, wherein X is A, T or S; and wherein n is 2-5 (SEQ ID NO: 17). In some embodiments, a peptide linker for use herein can have the amino acid sequence: GGGGTGGGGTGGGGT (SEQ ID NO: 18).

In some embodiments, compositions herein can include about 1 to about 10 linkers. In some examples, compositions herein can include about 1 to about 2 peptide linkers. In some examples, compositions herein do not have a linker. In some examples, a linker (e.g., a peptide linker) can be inserted at any amino acid residue of a protein, protein domain, peptide, peptide fragment, or polypeptide sequence disclosed herein. In some examples, a linker (e.g., a peptide linker) can be inserted at a C terminal end, at a N terminal end, or at both the C terminal and N terminal ends of a protein, protein domain, peptide, peptide fragment, or polypeptide sequence disclosed herein. In some examples, a linker (e.g., a peptide linker) can be inserted at a C terminal end, at a N terminal end, at both the C terminal and N terminal ends, and/or at any amino acid residue of any of the peptides having an amino acid sequence for ubiquitin as disclosed herein.

### (e) Biotin-Binding Proteins and Biotin-Acceptor Peptides

Biotinylation is the process of covalently attaching biotin to a biomolecule (e.g., a protein, nucleic acid or other biomolecule). In some embodiments, compositions herein can be biotinylated chemically, enzymatically, or a combination thereof.

Chemical biotinylation as used herein can employ one or more conjugation chemistries to yield a nonspecific biotinylation of amines, carboxylates, sulfhydryls and carbohydrates (e.g., NHS-coupling gives biotinylation of any primary amines in a protein such as those disclosed herein). In some examples, chemical biotinylation reagents can have one or more reactive groups attached to the valeric acid side chain of a biotin. In some examples, compositions herein can have one or more biotin binding proteins. Non-limiting examples of biotin binding proteins include avidin, deglycosylated avidin, native streptavidin, and recombinant streptavidin. The phrase “recombinant streptavidin” includes monovalent, divalent, trivalent, or tetravalent streptavidin, as well as truncated variants that comprise the 3-barrel structure characteristic of streptavidin. Truncated streptavidins are known in the art; see for example Sano et al. J. Biol. Chem 270: p. 28201 (1995), hereby incorporated by reference in its entirety.

A skilled artisan will be familiar with the numerous different biotinylation reagents and procedures that are well known in the art, in some cases commercially available, and suitable for the present disclosure. Biotin may be incorporated into or attached to a wide diversity of compounds, including, but not limited to proteins, peptides, nucleotides, carbohydrates, and polysaccharides. For example, proteins and peptides can be biotinylated on a free amine, sulfhydryl and/or carboxy group using an appropriate biotin derivative (e.g., N-hydroxysuccinimide ester (NHS-ester) of biotin, 3-(N-maleimidopropionyl) biocytin or iodoacetyl-LC biotin, or biocytin hydrazide, respectively). Carbohydrates or glycoproteins are easily biotinylated by using biotin-LC-hydrazide or biocytin hydrazide, after the vicinal hydroxyl group of the sugar has been oxidized to an aldehyde. Nucleic acid biotinylation can be accomplished with several different procedures, including introduction of biotinylated nucleotides using nick translation or random priming, or chemical labeling of aliphatic primary amines on nucleotide or modified nucleotide bases. Biotin binding proteins suitable for use herein may be recombinantly produced, purified from naturally producing organisms, or commercially acquired. Like biotin and its derivatives, biotin binding proteins may be modified with peptides, nucleic acids and/or carbohydrates following standard chemical procedures known to one skilled in the art. In some examples, one or more biotin molecules can be conjugated to a protein, protein domain, peptide, peptide fragment, or polypeptide sequence disclosed herein.

Enzymatic biotinylation as used herein can result in biotinylation of a specific lysine within a certain sequence by a biotin protein ligase (BPL). In some examples, compositions herein can be biotinylated by any enzyme that catalyzes coupling of biotin to one or more of the proteins, protein domains, peptides, peptide fragments, and polypeptide sequences disclosed herein. In some examples, an enzyme that catalyzes coupling of biotin suitable for use herein can be a biotin protein ligase (BPL). In some examples, a BPL suitable for use herein can be an E. coli biotin ligase (BirA). Bir A polypeptides and nucleic acids encoding BirA are described in U.S. Pat. Nos. 6,255,075 and 5,723,584 which are hereby incorporated in their entirety by reference. In some examples, a BPL (e.g., BirA) suitable for use herein can include modified and mutated forms of the BPL, in any host cell (e.g., the use of E. coli BirA in S. cerevisiae). In some embodiments, a BPL (e.g., BirA) suitable for use herein can be activated by lowering or increasing the temperature to a specific level.

In various embodiments, compositions herein can have at least one biotin-acceptor peptide. As used herein, a biotin-acceptor peptide can refer to a peptide having at least one biotinylation motif. Examples of biotinylation motifs suitable for use herein can include, but are not limited to, GLNDIFEAQKIEWHE (SEQ ID NO: 19), ALNDIFEAQKIEWHA (SEQ ID NO: 20), MAGGLNDIFEAQKIEWHEDTGGS (SEQ ID NO: 21), and LHHILDAQKMVWNHR (SEQ ID NO: 22). In some examples, biotinylation motifs suitable for use herein can be an AviTag which has SEQ ID NO: 19. In some examples, a biotin-acceptor peptide for use herein can be at least one biotin-acceptor peptide comprises 10 to 400 amino acids. In some examples, a biotin-acceptor peptide for use herein can be a biotin carboxyl carrier protein (BCCP) or a variant thereof. In certain embodiments, one or more biotin-acceptor peptides can be fused to an N terminal end a C terminal end and/or inserted in-frame into a coding sequence of any of the proteins, protein domains, peptides, peptide fragments, or polypeptide sequences disclosed herein.

### (f) Chemical Handles

In some embodiments, a target biomolecule used herein can be modified with at least one chemical handle. The term, “chemical handle,” as used herein, refers to a functional group that is capable of undergoing a click reaction, a 1,3-dipolar cycloaddition, and/or a Staudinger ligation. The skilled artisan can appreciate that methods of modifying a target biomolecule with a chemical handle can vary according to the type of chemical handle selected. Non-limiting chemical handles can include alkyne-reactive moieties, such as azide; and azide-reactive moieties, such as alkynes, including, but not limited to, terminal alkynes and activated alkynes; and phosphines, including, but not limited to, a triarylphosphine; and the like. In some embodiments, selection of a chemical handle can be dependent on the self-labeling tag used in the compositions disclosed herein. As used herein, a “biomolecule derivative” refers to a target biomolecule having at least one chemical handle. Addition of a chemical handle to a target biomolecule of interest can couple a small molecule to at least one of the self-labeling protein tags of the compositions disclosed herein. In some examples, a biomolecule derivative can be small molecule modified with at least one chemical handle. In some examples, a small molecule may be conjugated to at least one chemical handle. In some examples, a small molecule (e.g. biomolecule derivative) for use herein can be conjugated to benzylguanine (BG), chloropyrimidines (CLP), and the like. In some exemplary examples, a biomolecule derivative for use herein can be a small molecule conjugated to CLP.

### (g) Biomolecule Sensors

In various embodiments, compositions herein can be biomolecule sensors. In some embodiments, biomolecule sensors disclosed herein can have at least one protein, protein domain, peptide, peptide fragment, polypeptide sequence, or a combination thereof as disclosed herein. In certain embodiments, at least one biomolecule sensor disclosed herein can be included in a system for proximity-based labeling of a biomolecule.

In some embodiments, biomolecule sensors herein can have at least one peptide having an amino acid sequence for ubiquitin as described herein. In some embodiments, biomolecule sensors herein can have at least one tag as described herein. In some examples, biomolecule sensors herein can have at least one peptide tag as described herein. In some examples, biomolecule sensors herein can have at least one epitope tag as described herein. In some examples, biomolecule sensors herein can have at least one self-labeling protein tag as described herein. In some embodiments, biomolecule sensors herein can have at least one linker as described herein. In some embodiments, biomolecule sensors herein can have at least two linkers as described herein. In some embodiments, biomolecule sensors herein can have at least one protein, protein domain, peptide, peptide fragment, polypeptide sequence, or a combination thereof corresponding to a protein belonging to a SidE-family of ubiquitin (Ub) ligases or a homolog thereof as described herein. In some examples, biomolecule sensors herein can have at least one peptide described herein as having an ADP-Ribosyltransferase (ART) domain of a SidE-ligase protein, at least one ortholog of SidE-ligase protein, or at least one paralog of a SidE-ligase protein. In some examples, biomolecule sensors herein can have at least one peptide described herein as having a phosphodiesterase (PDE) domain of a SidE-ligase protein, at least one ortholog of SidE-ligase protein, or at least one paralog of a SidE-ligase protein.

In some examples, biomolecule sensors herein can have at least one construct having at least one protein, protein domain, peptide, peptide fragment, polypeptide sequence, or a combination thereof as disclosed herein. A construct as used herein can refer to a recombinant protein generated from an expression vector containing the coding sequence of a recombinant protein. In some embodiments, a construct herein can have at least one peptide having an amino acid sequence for ubiquitin as described herein, at least one self-labeling protein tag as described herein, at least one linker as described herein, or a combination thereof. In some embodiments, a construct herein can have at least one peptide having an amino acid sequence for ubiquitin as described herein, at least one target protein as described herein, at least one linker as described herein, or a combination thereof. As used herein, a “target protein” can be a protein, a protein fragment, a peptide, or a polypeptide sequence of a protein used to identify a protein-protein interaction in the methods described herein. In some embodiments, a construct herein can further have at least one epitope tag disclosed herein. In some embodiments, a construct herein can further have at least one biotin-acceptor peptide disclosed herein. In some examples, a construct herein can be a recombinant protein having the following in order of N terminus to C terminus: a first epitope tag, a peptide having a genetically modified sequence for ubiquitin, a second epitope tag, a linker, a third epitope tag, a self-labeling protein tag, and a biotin-acceptor peptide. In some exemplary examples, construct can be a recombinant protein having the following in order of N terminus to C terminus: a His tag, a peptide having a genetically modified sequence for ubiquitin selected from SEQ ID NOs: 11-16, an HA tag, a linker having an amino acid sequence corresponding to SEQ ID NO: 18, a Flag tag, a SNAP tag, and a biotin-acceptor peptide having an amino acid sequence corresponding to SEQ IN NO: 19 (Avi tag).

In some embodiments, the biomolecule sensor composition disclosed herein may comprise three constructs which together have the formula (I):

Ub-T1-L-T2-S-A;

R; and

P (I),

wherein, Ub can be a peptide having a sequence for ubiquitin; T1 and T1 can each be an optional epitope tag; L can be a linker; S can be a self-labeling protein tag; A can be a biotin-acceptor peptide; R can be a peptide having a ADP-Ribosyltransferase (ART) domain of a SidE-ligase protein, at least one ortholog of SidE-ligase protein, or at least one paralog of a SidE-ligase protein; and P can be a peptide having a phosphodiesterase (PDE) domain of a SidE-ligase protein, at least one ortholog of SidE-ligase protein, or at least one paralog of a SidE-ligase protein.

In certain embodiments, any of the constructs disclosed herein can be prepared by recombinant technology as exemplified below.

In certain embodiments, biomolecule sensors herein can be attached to at least one solid support. In some examples, a solid support suitable for use herein can be small beads, pellets, disks, chips, wafers, and the like. In some examples, biomolecule sensors herein can be attached to beads suitable for use herein. Non-limited examples of beads suitable for use herein can include agarose beads, streptavidin-coated beads, NeutrAvidin-coated beads, antibody-coated beads, paramagnetic beads, magnetic beads, electrostatic beads, electrically conducting beads, fluorescently labeled beads, colloidal beads, glass beads, semiconductor beads, and polymeric beads. One of skill in the art will appreciate that biomolecule sensors herein can be attached to beads using methods suitable to the selected bead type. In some examples, biomolecule sensors herein can be coupled onto beads either covalently with typical NHS-ester coupling procedures or with carboxy-groups on the surface of the beads. An alternative way can be to couple a His-tagged recombinant protein to the beads with a Ni-NTA surface. Another option can be the covalent coupling of a specific or Tag-specific antibody to a bead. In some examples, biomolecule sensors herein can be coupled in an indirect way to a bead (examples for tag-specific antibodies: anti-His-tag antibody, anti-glutathione-transferase antibody, anti-Maltose binding protein, and others). In some embodiments, beads as described can be combined with antibodies or other binders, like scaffold proteins. Non-limiting examples of binding proteins include anticalins, ancyrin repeat proteins, cysteine-knot proteins, nanobodies and the like. In other embodiments, beads can be functionalized with enzymes (e.g., phosphatases, kinases, lipases) or nucleotide sequences. In some examples, such nucleotide sequences can occur as primers, DNA or RNA fragments, binding molecules, like aptamers or as larger DNA or RNA structures. In some examples, functionalization of beads herein with, for example antibodies or other structures, can be achieved by covalent coupling by NHS-chemistry or by tags, which are captured by the corresponding affinity ligand, for example His-tag—Ni2+-NTA, GST-tag—glutathione, S-tag—S-protein.

### (h) Biomolecule Sensor Systems

The biomolecule sensors disclosed herein can be utilized in biomolecule sensor systems. As used herein, a “biomolecule sensor system” can include a combination any of the biomolecules, biomolecule derivatives, proteins, protein domains, peptides, peptide fragments, and polypeptide sequences disclosed herein that can be used to identify at least one biomolecule. In some embodiments, a biomolecule sensor system (e.g., “system”) can be used herein for proximity-based labeling of at least one biomolecule. Proximity-based labeling is an enzyme-catalyzed method that labels biomolecules proximal to a protein of interest. In some embodiments, a biomolecule sensor system (e.g., “system”) can be used herein for proximity-based labeling of at least one biomolecule in a sample. In some embodiments, a biomolecule sensor system (e.g., “system”) can be used herein for proximity-based labeling to identify at least one biomolecule in a sample wherein the presence of the biomolecule is unknown.

In some embodiments, biomolecule sensor systems herein may include: a construct comprising a genetically modified ubiquitin (Ub), a self-labeling protein tag, a linker, and optionally, one or more tags; at least one biomolecule derivative; and at least two protein domains of a SidE-ligase protein, an ortholog of SidE-ligase protein, or a paralog of a SidE-ligase protein. As disclosed herein, a “biomolecule derivative” refers to a target biomolecule having at least one chemical handle. In some embodiments, biomolecule sensor systems herein having: a construct comprising a genetically modified ubiquitin (Ub), a self-labeling protein tag, a linker, and optionally, one or more tags; a biomolecule derivative; and at least two protein domains of a SidE-ligase protein, an ortholog of SidE-ligase protein, or a paralog of a SidE-ligase protein, can be used herein for proximity-based labeling of at least one biomolecule. In some examples, biomolecule sensor systems herein having: a construct comprising a genetically modified ubiquitin (Ub), a self-labeling protein tag, a linker, and optionally, one or more tags; a biomolecule derivative; and at least two protein domains of a SidE-ligase protein, an ortholog of SidE-ligase protein, or a paralog of a SidE-ligase protein, can be used herein for proximity-based labeling of at least one small molecule.

In some embodiments, biomolecule sensor systems herein may include: a construct comprising a genetically modified ubiquitin (Ub), a target protein, a linker, and optionally, one or more tags; and at least two protein domains of a SidE-ligase protein, an ortholog of SidE-ligase protein, or a paralog of a SidE-ligase protein. As disclosed herein, a “target protein” refers to a protein of interest having or suspected of having at least one interaction with at least one other protein. In some embodiments, biomolecule sensor systems herein having a construct comprising a genetically modified ubiquitin (Ub), a target protein, a linker, and optionally, one or more tags, and at least two protein domains of a SidE-ligase protein, an ortholog of SidE-ligase protein, or a paralog of a SidE-ligase protein, can be used herein for proximity-based labeling of at least one biomolecule. In some examples, biomolecule sensor systems herein having a construct comprising a genetically modified ubiquitin (Ub), a target protein, a linker, and optionally, one or more tags, and at least two protein domains of a SidE-ligase protein, an ortholog of SidE-ligase protein, or a paralog of a SidE-ligase protein, can be used herein for proximity-based labeling of at least one protein.

### III. Methods of Preparing Compositions

In various embodiments, any of the proteins, protein domains, peptides, peptide fragments, and polypeptide sequences disclosed herein can be produced via, e.g., conventional recombinant technology. In some examples, DNA encoding any of the proteins, protein domains, peptides, peptide fragments, or polypeptide sequences disclosed herein can be readily isolated and sequenced using conventional procedures (e.g., by using oligonucleotide probes that are capable of binding specifically to genes encoding a polypeptide sequence). Once isolated, the DNA may be placed into one or more expression vectors, which are then transfected into host cells such as E. coli cells, simian COS cells, Chinese hamster ovary (CHO) cells, Human Embryotic Kidney (HEK) 293 cells or myeloma cells that do not otherwise produce the proteins, protein domains, peptides, peptide fragments, and polypeptide sequences disclosed herein. The DNA can then be modified accordingly for generating any of the compositions disclosed herein.

In some examples, any of the proteins, protein domains, peptides, peptide fragments, and polypeptide sequences disclosed herein can be prepared by recombinant technology as exemplified below.

Nucleic acids encoding any of the proteins, protein domains, peptides, peptide fragments, and polypeptide sequences disclosed herein can be cloned into one expression vector, each nucleotide sequence being in operable linkage to a suitable promoter. In some examples, each of the nucleotide sequences encoding any of the proteins, protein domains, peptides, peptide fragments, and polypeptide sequences disclosed herein can be in operable linkage to a distinct prompter. Alternatively, the nucleotide sequences encoding any of the proteins, protein domains, peptides, peptide fragments, and polypeptide sequences disclosed herein can be in operable linkage with a single promoter, such that one or more proteins are expressed from the same promoter. When necessary, an internal ribosomal entry site (IRES) can be inserted between protein encoding sequences.

In some examples, the nucleotide sequences encoding any of the proteins, protein domains, peptides, peptide fragments, and polypeptide sequences disclosed herein can be cloned into two vectors, which can be introduced into the same or different cells. When any of the proteins, protein domains, peptides, peptide fragments, and polypeptide sequences disclosed herein are expressed in different cells, each of them can be isolated from the host cells expressing such and the isolated proteins can be mixed and incubated under suitable conditions allowing, for example, methods of detecting protein interactions as disclosed herein.

Generally, a nucleic acid sequence encoding one or all of the proteins, protein domains, peptides, peptide fragments, and polypeptide sequences disclosed herein can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the decoy fusion proteins.

A variety of promoters can be used for expression of any of the proteins, protein domains, peptides, peptide fragments, and polypeptide sequences disclosed herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters can include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al,, Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, vectors used herein can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the proteins, protein domains, peptides, peptide fragments, and polypeptide sequences disclosed herein may be introduced into suitable host cells for producing the any of the proteins, protein domains, peptides, peptide fragments, or polypeptide sequences. The host cells can be cultured under suitable conditions for expression of any of the proteins, protein domains, peptides, peptide fragments, and polypeptide sequences disclosed herein. Such proteins, protein domains, peptides, peptide fragments, and/or polypeptide sequences can be recovered by the cultured cells (e.g., from the cells or the culture supernatant) via a conventional method, e.g., affinity purification. If necessary, any of the proteins, protein domains, peptides, peptide fragments, or polypeptide sequences disclosed herein can be incubated under suitable conditions for a suitable period of time allowing for production of the proteins, protein domains, peptides, peptide fragments.

In some embodiments, methods for preparing any of the proteins, protein domains, peptides, peptide fragments, and polypeptide sequences disclosed herein described herein can involve a recombinant expression vector that encodes all components of the any of the proteins, protein domains, peptides, peptide fragments, or polypeptide sequences also disclosed herein. The recombinant expression vector can be introduced into a suitable host cell (e.g., a HEK293T cell or a dhfr-CHO cell) by a conventional method, e.g., calcium phosphate-mediated transfection. Positive transformant host cells can be selected and cultured under suitable conditions allowing for the expression of any of the proteins, protein domains, peptides, peptide fragments, and polypeptide sequences disclosed herein which can be recovered from the cells or from the culture medium. When necessary, any of the proteins, protein domains, peptides, or peptide fragments recovered from the host cells can be incubated under suitable conditions allowing for the formation of decoy fusion protein homodimers.

Standard molecular biology techniques are used to prepare the recombinant expression vector, transfect the host cells, select for transformants, culture the host cells and recovery of the decoy fusion proteins from the culture medium. For example, some proteins, protein domains, peptides, or peptide fragments disclosed herein can be isolated by affinity chromatography with a Protein A or Protein G coupled matrix.

### IV. Methods of Use

The present disclosure provides methods for detecting at least one protein interaction with at least one target biomolecule using compositions disclosed herein. In certain embodiments, methods herein can be used to detect at least one protein interaction with at least one target biomolecule de novo. As used herein, “de novo” refers to methods of identifying at least one protein interaction with at least one target biomolecule wherein the possibly of the interaction was not known prior to performing the method. In some examples, methods herein can be used to detect at least one protein interaction with at least one target biomolecule de novo from raw mass spectrometry data. In certain embodiments, methods herein can be used to detect at least one protein interaction with at least one target small molecule. In certain embodiments, methods herein can be used to detect at least one protein interaction with at least one target protein.

### (a) Proximity-Based Labeling of Protein-Small Molecule Interactions

In certain embodiments, methods using any of the compositions disclosed herein can be used to detect at least one a protein-small molecule interaction with at least one target small molecule (e.g., proximity-based labeling). In some examples, a target small molecule can be modified with at least one chemical handle. In some embodiments, a target small molecule can be modified by conjugation to benzylguanine (BG), chloropyrimidine (CLP), or a combination thereof.

In certain embodiments, a first reaction of a method herein can be ADP ribosylation of a construct disclosed herein by at least one ADP-Ribosyltransferase (ART) domain of a SidE-ligase protein or its homolog. In some examples, a ribosylated construct can have at minimum at least one a peptide having a genetically modified sequence for ubiquitin and at least one self-labeling protein tag. In some examples, an ART domain of a SidE-ligase protein or its homolog can use NAD+ as a cofactor to transfer at least one ADP-ribose to a construct herein having a genetically modified ubiquitin peptide. Nicotinamide adenine dinucleotide (NAD) is a cofactor central to metabolism that can exist in two forms: an oxidized form (NAD+) and a reduced form (NADH where “H” is for hydrogen). In some examples, an ART domain of a SidE-ligase protein or its homolog can transfer at least one ADP-ribose to one or more amino acid residues in the construct having a genetically modified ubiquitin peptide. In some examples, an ART domain of a SidE-ligase protein or its homolog can transfer at least one ADP-ribose to the amino acid residue Arg 42 in the construct having a genetically modified ubiquitin peptide.

In certain embodiments, a second reaction of a method herein can be association of at least one target small molecule to a construct disclosed herein. In some examples, a small molecule can be cross-linked to a construct herein. In some examples, at least one target small molecule can be conjugated to a construct disclosed herein. In some examples, at least one target small molecule can be conjugated to a ribosylated construct having, at minimum, at least one a peptide having a genetically modified sequence for ubiquitin and at least one self-labeling protein tag. In some examples, target small molecule can be a target small molecule modified with at least one chemical handle conjugated to a ribosylated construct herein at the least one self-labeling protein tag within the construct. In some examples, a small molecule modified with CLP can be conjugated to a ribosylated construct herein having a SNAP-tag.

In certain embodiments, a ribosylated construct having at least one target small molecule attached can be added to a sample. A “sample” as used herein can be any material suspected of having or having at least one protein that will interact with a target biomolecule (e.g., a target small molecule and/or a target protein). Samples analyzed by the methods disclosed herein can be obtained from any source known to those skilled in the art. In some examples, a sample for use herein can be a microbiome sample. In some non-limiting embodiments, a microbiome sample can be obtained from soil, air, water (including, without limitation, marine water, fresh water, and rain water), sediment, oil, and combinations thereof. In other non-limiting embodiments, a microbiome sample can be obtained from a subject selected from a protozoa, an animal (e.g., a mammal, e.g., human), or a plant. In some other examples, a sample for use herein can be from a subject. The term “subject” as used herein refers to an animal, including but not limited to a mammal including a human and a non-human primate (for example, a monkey or great ape), a cow, a pig, a cat, a dog, a rat, a mouse, a horse, a goat, a rabbit, a sheep, a hamster, a guinea pig). In some embodiments, a subject can be at a genetic risk for development a disease. Non-limiting examples of such diseases include digestive system diseases, cardiovascular diseases, neurological diseases, obesity, diabetes, and cancers. In other embodiments, the subject may be at a risk of having, or have a bacterial infection (e.g., pneumonia) infection and/or a viral infection (e.g., HIV-AIDS, influenza, coronavirus). In various embodiments, a sample obtained from an animal subject can be a body fluid. In other embodiments, a sample obtained from an animal subject can be a tissue sample. Non-limiting samples obtained from an animal subject can include tooth, perspiration, fingernail, skin, hair, feces, urine, semen, mucus, saliva, sputum, blood, serum, plasma, bone marrow, tissue biopsy, liquid biopsy, and the like. In some embodiments, a sample can be a cell lysate. In some examples, a cell lysate can be prepared from an established cell line, a primary cell line, or a cell isolated from at least one subject.

In certain embodiments, a ribosylated construct having at least one target small molecule attached can be added to a sample and incubated. A time of incubation can be optimized depending on source of sample, known efficiency of the target small molecule, components and buffers used in the method, and the like. In some embodiments, about 1 nM to about 1 M of a ribosylated construct having at least one target small molecule attached can be added to a sample for incubation. In some embodiments, about 1 nM, about 100 nM, about 200 nM, about 300 nM, about 400 nM, about 500 nM, about 600 nM, about 700 nM, about 800 nM, about 900 nM, about 1 μM, about 100 μM, about 200 μM, about 300 μM, about 400 μM, about 500 μM, about 600 μM, about 700 μM, about 800 μM, about 900 μM, about 1 mM, about 100 mM, about 200 mM, about 300 mM, about 400 mM, about 500 mM, about 600 mM, about 700 mM, about 800 mM, about 900 mM, or about 1 M of a ribosylated construct having at least one target small molecule attached can be added to a sample for incubation. In some examples, about 1 mM to about 500 mM, about 25 mM to about 250 mM, or about 50 mM to about 100 mM of ribosylated construct having at least one target small molecule attached can be added to a sample for incubation.

In certain embodiments, a third reaction of a method herein can be a covalent linkage between a ribosylated construct having at least one attached target small molecule and at least protein within a sample that interacts with the attached target small molecule, wherein the covalent linkage is formed by at least one PDE domain of a SidE-ligase protein or its homolog. In certain methods, at least one PDE domain of a SidE-ligase protein or its homolog can be added to the incubation of a sample with at least one ribosylated construct having at least one attached target small molecule. In some embodiments, PDE domain of a SidE-ligase protein or its homolog can link the ribosylated construct having at least one attached target small molecule to at least one interacting protein in the sample by hydrolyzing the phosphodiester bond to generate phosphoribosylated Ub. In some embodiments, PDE domain of a SidE-ligase protein or its homolog can link the ribosylated construct having at least one attached target small molecule to at least one interacting protein in the sample by linking the Ub an interacting protein by forming a serine-phosphoribosyl-Ub linkage.

In certain embodiments, about 1 ng to about 1 g of at least one PDE domain of a SidE-ligase protein or its homolog can be added to the sample incubated with at least one ribosylated construct having at least one attached target small molecule. In some embodiments, about 1 ng, about 100 ng, about 200 ng, about 300 ng, about 400 ng, about 500 ng, about 600 ng, about 700 ng, about 800 ng, about 900 ng, about 1 μg, about 100 μg, about 200 μg, about 300 μg, about 400 μg, about 500 μg, about 600 μg. about 700 μg, about 800 μg, about 900 μg, about 1 mg, about 100 mg, about 200 mg, about 300 mg, about 400 mg, about 500 mg, about 600 mg, about 700 mg, about 800 mg, about 900 mg, or about 1 M of at least one PDE domain of a SidE-ligase protein or its homolog can be added to the sample incubated with at least one ribosylated construct having at least one attached target small molecule. In some examples, about 1 μg to about 500 μg, about 25 μg to about 250 μg, or about 50 μg to about 100 μg of at least one PDE domain of a SidE-ligase protein or its homolog can be added to the sample incubated with at least one ribosylated construct having at least one attached target small molecule. In some examples, about 60 μg of at least one PDE domain of a SidE-ligase protein or its homolog can be added to the sample incubated with at least one ribosylated construct having at least one attached target small molecule.

### (b) Proximity-Based Labeling of Protein-Protein Interactions

In certain embodiments, methods using any of the compositions disclosed herein can be used to detect at least one a protein-protein interaction with at least one target protein.

In certain embodiments, a first reaction of a method herein can be ADP ribosylation of a construct disclosed herein by at least one ADP-Ribosyltransferase (ART) domain of a SidE-ligase protein or its homolog. In some examples, a ribosylated construct can have at minimum at least one a peptide having a genetically modified sequence for ubiquitin and a target protein. In some examples, a target protein can be fused to a genetically modified sequence for ubiquitin using recombinant expression. In some examples, a target protein can be fused to a genetically modified ubiquitin peptide using one or more protein-conjugation methods known in the art. In some examples, a target protein can be a peptide encompassing an amino acid sequence that has or is suspected of having a binding motif for one or more protein interactions. In some examples, an ART domain of a SidE-ligase protein or its homolog can use NAD+ as a cofactor to transfer at least one ADP-ribose to a construct herein having a genetically modified ubiquitin peptide. In some examples, an ART domain of a SidE-ligase protein or its homolog can transfer at least one ADP-ribose to one or more amino acid residues in the construct having a genetically modified ubiquitin peptide. In some examples, an ART domain of a SidE-ligase protein or its homolog can transfer at least one ADP-ribose to the amino acid residue Arg 42 in the construct having a genetically modified ubiquitin peptide.

In certain embodiments, a ribosylated construct having at least one target protein attached can be added to a sample and incubated according to methods disclosed herein. In some embodiments, about 1 nM to about 1 M of a ribosylated construct having at least one target protein attached can be added to a sample for incubation. In some embodiments, about 1 nM, about 100 nM, about 200 nM, about 300 nM, about 400 nM, about 500 nM, about 600 nM, about 700 nM, about 800 nM, about 900 nM, about 1 μM, about 100 μM, about 200 μM, about 300 μM, about 400 μM, about 500 μM, about 600 μM, about 700 μM, about 800 μM, about 900 μM, about 1 mM, about 100 mM, about 200 mM, about 300 mM, about 400 mM, about 500 mM, about 600 mM, about 700 mM, about 800 mM, about 900 mM, or about 1 M of a ribosylated construct having at least one target protein attached can be added to a sample for incubation. In some examples, about 1 mM to about 500 mM, about 25 mM to about 250 mM, or about 50 mM to about 100 mM of ribosylated construct having at least one target protein attached can be added to a sample for incubation.

In certain embodiments, a third reaction of a method herein can be a covalent linkage between a ribosylated construct having at least one attached target protein and at least one protein from within the sample that interacts with the attached target protein. In some aspects, the covalent linkage can be formed by at least one protein, protein domain, peptide, peptide fragment, polypeptide sequence, or a combination thereof corresponding to a protein belonging to a SidE-family of ubiquitin (Ub) ligases. In some other aspects, the covalent linkage can be formed by one or more of the functionally redundant proteins (SdeA, SdeB, SdeC and/or SidE) comprising the SidE-family. In some examples, the covalent linkage can be formed by one or more full length SidE effectors selected from full-length SdeA, full-length SdeB, full-length SdeC, full-length SidE, and full-length homologs and/or full-length paralogs thereof. In some other examples, the covalent linkage can be formed by one or more protein domains of SidE effectors selected from any of the protein domains of SdeA, the protein domains of SdeB, the protein domains of SdeC, the protein domains of SidE, and the protein domains of homologs and/or paralogs thereof. In some aspects, the covalent linkage can be formed by at least one PDE domain of a SidE-ligase family protein (e.g., at least one PDE domain of SdeA, SdeB, SdeC, SidE, homologs, and paralogs thereof). In some aspects, the covalent linkage can be formed by at least one PDE domain of a SidE-ligase protein or its homolog.

In certain methods, at least one full length SidE effector, one PDE domain of a SidE-ligase protein, their homologs and/or paralogs can be added to the sample incubated with at least one ribosylated construct having at least one attached target protein. In some embodiments, at least one full length SidE effector, one PDE domain of a SidE-ligase protein, their homologs and/or paralogs can link the ribosylated construct having at least one attached target protein by methods described herein.

In certain embodiments, about 1 ng to about 1 g of at least one full length SidE effector protein or its paralog can be added to the sample incubated with at least one ribosylated construct having at least one attached target protein. In some embodiments, about 1 ng, about 100 ng, about 200 ng, about 300 ng, about 400 ng, about 500 ng, about 600 ng, about 700 ng, about 800 ng, about 900 ng, about 1 μg, about 100 μg, about 200 μg, about 300 μg, about 400 μg, about 500 μg, about 600 μg, about 700 μg, about 800 μg, about 900 μg, about 1 mg, about 100 mg, about 200 mg, about 300 mg, about 400 mg, about 500 mg, about 600 mg, about 700 mg, about 800 mg, about 900 mg, or about 1 M of at least one full length SidE effector protein or its paralog can be added to the sample incubated with at least one ribosylated construct having at least one attached target protein. In some examples, about 1 μg to about 500 μg, about 25 μg to about 250 μg, or about 50 μg to about 100 μg of at least full length SidE effector protein or its paralog can be added to the sample incubated with at least one ribosylated construct having at least one attached target protein. In some examples, about 60 μg of at least one full length SidE effector protein or its paralog can be added to the sample incubated with at least one ribosylated construct having at least one attached target protein.

In certain embodiments, about 1 ng to about 1 g of at least one PDE domain of a SidE-ligase protein or its homolog can be added to the sample incubated with at least one ribosylated construct having at least one attached target protein. In some embodiments, about 1 ng, about 100 ng, about 200 ng, about 300 ng, about 400 ng, about 500 ng, about 600 ng, about 700 ng, about 800 ng, about 900 ng, about 1 μg, about 100 μg, about 200 μg, about 300 μg, about 400 μg, about 500 μg, about 600 μg, about 700 μg, about 800 μg, about 900 μg, about 1 mg, about 100 mg, about 200 mg, about 300 mg, about 400 mg, about 500 mg, about 600 mg, about 700 mg, about 800 mg, about 900 mg, or about 1 M of at least one PDE domain of a SidE-ligase protein or its homolog can be added to the sample incubated with at least one ribosylated construct having at least one attached target protein. In some examples, about 1 μg to about 500 μg, about 25 μg to about 250 μg, or about 50 μg to about 100 μg of at least one PDE domain of a SidE-ligase protein or its homolog can be added to the sample incubated with at least one ribosylated construct having at least one attached target protein. In some examples, about 60 μg of at least one PDE domain of a SidE-ligase protein or its homolog can be added to the sample incubated with at least one ribosylated construct having at least one attached target protein.

### (c) Purification and Analysis

In certain embodiments, methods used to detect at least one protein interaction with at least one target biomolecule (e.g., target small molecule, target protein) can include purification of a ribosylated construct having at least one attached target biomolecule after a covalent linkage is formed with an interacting protein. In some embodiments, purification can be performed using standard chromatography methods. In some embodiments, where a construct used in the method further included at least one epitope tag, purification can be performed using standard immuno-pull down methods. In some embodiments, where a construct used in the methods further included at least one biotin-acceptor peptide, purification can be performed using standard avidin-pull down methods. In some examples, where a construct used in the methods further included at least one biotin-acceptor peptide, purification can be performed using avidin-beads.

In certain embodiments, methods used to detect at least one protein interaction with at least one target biomolecule (e.g., target small molecule, target protein) can include any immunoassay known in the art, for example an ELISA assay or Western blotting. An immunoassay can be performed before purification or after purification of the ribosylated construct having at least one attached target biomolecule after a covalent linkage is formed with an interacting protein.

In certain embodiments, methods used to detect at least one protein interaction with at least one target biomolecule (e.g., target small molecule, target protein) can include mass spectrometry. In some examples, methods of detecting a protein interaction with at least one target biomolecule can involve digestion of the sample having a ribosylated construct having at least one attached target biomolecule after a covalent linkage is formed with an interacting protein before the construct is purified from the sample. In some examples, the digestion method is performed in the presence of beads. In some examples, the digestion method is performed in the presence of avidin-beads.

In some embodiments, methods of detecting a protein interaction with at least one target biomolecule can involve digestion of the sample having a ribosylated construct having at least one attached target biomolecule after a covalent linkage is formed with an interacting protein after the construct is purified from the sample. In some examples, methods of detecting a protein interaction with at least one target biomolecule can not involve digestion of the sample having a ribosylated construct having at least one attached target biomolecule after a covalent linkage is formed with an interacting protein before the construct is purified from the sample.

In some embodiments, methods herein can further include one or more additional processes for preparing a digested, purified construct prior to detection of at least one protein interaction with at least one target biomolecule. In some embodiments, an additional process herein can include having one or more reagents removed before or after digestion and proteins reduced and/or alkylated using methods known in the art. In some examples, a digested, purified construct herein may be further treated with one or more chaotropic agents (e.g., Urea and/or guanidine hydrochloride (GuHCl)). In some embodiments, a digested, purified construct herein may be further digested with one or more proteolytic enzymes (e.g., trypsin, Glu-C enzyme, Asp-N enzyme, pepsin, tryp-N enzyme, elastase, Arg-C enzyme, chymotrypsin). In some embodiments, a digested, purified construct herein may be further subjected to one or more chemical digestions (e.g., with cyanogen bromide and/or hydroxylamine). In some embodiments, a digested, purified construct herein may be desalted (e.g., by reversed phase). In some examples, a digested, purified construct herein may be treated with one or more deubiquitinases. Non-limiting examples of deubiquitinases suitable for such methods can be DupA, DupB, and the like. In some embodiments, one or more reagents may be removed by acetone precipitation, trizol extraction, ion exchange chromatography, or other solid phase extraction techniques commonly used in the field.

In certain aspects, a digested, purified construct can be subjected to mass spectrometry to detect at least one protein interaction with at least one target biomolecule. In certain aspects, an undigested, purified construct can be subjected to mass spectrometry to detect at least one protein interaction with at least one target biomolecule. Non-limited examples of mass spectrometry that can be used herein can include liquid chromatography-tandem mass spectrometry (LC-MS/MS) gas phase ion spectrometry, laser desorption mass spectrometry, tandem mass spectrometry, electrospray mass spectrometry, Surface-Enhanced Laser Desorption/Ionization (“SELDI”) mass spectrometry, Matrix-Assisted Laser Desorption Ionization-time of Flight Mass Spectrometry (MALDI-TOF MS), Edman degradation, or any combination thereof.

### IV. Kits

The present disclosure provides kits for performing any of the methods disclosed herein. In certain embodiments, kits herein can be used to prepare at least one of the compositions disclosed herein. In some examples, kits herein can be used to generate one or more of the biomolecule sensors disclosed herein. In some examples, kits herein can be used to generate any of the constructs disclosed herein. In some examples, kits herein can contain any of the materials needed to generate recombinant constructs disclosed herein, wherein the materials can be any of those known to the skilled artisan to be useful in standard molecular biology protocols such as, but not limited to, expression vectors, restriction enzymes, PCR buffers and enzymes, resins, and the like. In some embodiments, kits herein can further include instructions on how to generate any of the compositions disclosed herein (e.g., biomolecule sensors, constructs, systems, etc.).

In certain embodiments, kits herein can be used to perform methods of detecting a protein interaction with a target biomolecule as disclosed herein. In some examples, kits can have components needed to generate any of the compositions disclosed herein (e.g., biomolecule sensors, constructs, systems, etc.) used in the methods described above. In some examples, kits can have pre-paired compositions, at least one pre-paired component of a composition, or a combination thereof. In some embodiments, kits herein can have a construct disclosed herein, a peptide having an ADP-Ribosyltransferase (ART) domain of a SidE-ligase protein or its homolog, a peptide having a phosphodiesterase (PDE) domain of a SidE-ligase protein or its homolog, or a combination thereof. In some examples, a kit herein can have an isolated peptide of a SidE-ligase protein or its homolog having both an ART and a PDE domain, an isolated peptide having an ART domain of a SidE-ligase protein or its homolog, an isolated peptide having a PDE domain of a SidE-ligase protein or its homolog, or a combination thereof. In some examples, kit herein can further have at least one target biomolecule, at least one a target biomolecule derivative, or a combination thereof. In some examples, a kit can have at least one target biomolecule, at least one chemical handle, and instructions on how to prepare a target biomolecule derivative. In some embodiments, kits herein can have instructions on how to perform any of the methods of detecting a protein interaction as disclosed herein.

In certain embodiments, kits herein can contain at least one solid support to be used in performing the methods disclosed herein. In some examples, kits herein can have beads suitable for performing the methods disclosed herein.

In certain embodiments, kits herein can include at least one container and/or a label or package insert(s) on or associated with the container. In some embodiments, the invention provides articles of manufacture comprising contents of the kits described above. The kits of this invention can be in suitable packaging. Suitable packaging includes, but is not limited to, vials, bottles, jars, flexible packaging (e.g., sealed Mylar or plastic bags), and the like. Any instructions included in kits herein can be written instructions on a label or package insert (e.g., a paper sheet included in the kit), but machine-readable instructions (e.g., instructions carried on a magnetic or optical storage disk) are also acceptable.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Introduction to Examples 1-3

Small molecule drugs are widely used in the treatment of a large variety of diseases. Despite the efficacy of these small molecules, a surprising number of them lack known mechanisms of actions or identified targets. Identifying the targets of these orphan compounds is nontrivial and presents a substantial obstacle in drug discovery.

Although several strategies exist and have been successfully employed for target identification of small molecule drugs, each approach has drawbacks that present major challenges in target validation. For example, biochemical methods are useful in that they require direct interactions, but often suffer from high background and require pre-existing knowledge to narrow down candidate hits. The covalent labeling of a small molecule with biotin and subsequent immobilization of the biotin-tagged small molecule to avidin resin is a common technique. However, for this method to be successful, strong drug-target binding interactions are required. Moreover, this strategy suffers from a lack of stringent washing conditions and thus, a high background of non-specific interactors. Alternative target identification methods utilize activity-based and photoreactive crosslinking probes coupled to MS/MS. These methods require the ligand binding pocket of the target to contain a reactive amino acid for conjugation. This strategy is generally confined to enzymes, depends on the orientation of the ligand, and suffers from the high chemical reactivity of the probe resulting in high background. Thus, while the aforementioned methods may capture direct interactions, they all suffer from high background noise and require a priori knowledge to narrow down a large list of potential candidates.

Screen and clonal variation-based methods provide an unbiased approach to target identification. However, these methods do not prove direct interactions and are often hindered by noise, computational power, and large investment costs. The nature of CRISPR/RNAi screens limits them to non-essential gene targets, excluding a large pool of clinically efficacious drugs. Moreover, results from these screens are often noisy due to the off-target effects of the guides or the siRNA. Attempts to mitigate these effects introduces prohibitively large ultra-complex libraries that are generally not feasible when conducting whole genome screens. Forward genetics also falters in many situations. For example, screening for individual mutants is often problematical in cancers with non-diploid aberrant chromosomal features. It is also limited by mutational rates of the cell-lines, potentially leading to exponential time requirements and statistically improbable chances of generating appropriate clones. While both of these approaches are unbiased and require no prior knowledge, the high input costs of these protocols, as well as the level of complexity in many diseases limits its application.

Chemoinformatic approaches such as the Similarity Ensemble Approach (SEA) are a relatively cost-effective strategy that utilizes previously published literature and data to generate a list of most likely candidates. This approach holds promise for molecules that are similar to well-studied drugs with soundly annotated characteristics. However, if targets have poorly annotated ligands or do not report shared chemotypes, the SEA algorithm will fail to find an association. Additionally, much of the final identification requires extensive manual curation of existing literature, representing a time-consuming and rate-limiting step in the process.

The most promising method for quick, effective, and unbiased identification of direct interactors is the proximity labelling approach. Of the few known systems, each utilize enzymes (e.g., a biotin ligase, a peroxidase) to generate reactive biotin moieties that diffuse to neighboring proteins and conjugate to reactive residues. However, the large labeling radius of these systems introduces high levels of background, confounding final target validation. Although these systems have been used to direct protein-protein interactions, they have not been applied to small molecule target identification.

### Example 1. Generation of SidBait Constructs

The SidE-family of ubiquitin (Ub) ligases are type 4 secretion system effector proteins found in the Gram-negative human bacterial pathogen Legionella pneumophila, the causative agent of Legionnaires' disease in humans. The SidE-family consists of four functionally redundant proteins (SdeA, SdeB, SdeC and SidE) that contain an N-terminal deubiquitinase domain, an HD-like phosphodiesterase (PDE) domain, an ADP Ribosyltransferase (ART) domain and a C-terminal domain (CTD) containing a coiled coil region (FIG. 1A). The SidE effectors catalyze the covalent addition of Ub to serine (Ser) residues on host proteins independent of the cellular E1 and E2 Ub conjugating enzymes. The first step in this reaction requires the SidE ART domain, which binds cellular NAD+ and transfers an ADP ribose to an internal Arg residue on Ub (Arg42). ADP-ribosylated Ub acts as a substrate for the PDE domain, which hydrolyzes the phosphodiester bond in ADP ribose and, when a host protein is in the vicinity, it will become ubiquitinated on a Ser residue via a phosphoribose linkage (FIGS. 1B and 1C). Importantly, this reaction does not require the C-terminal diglycine motif or any Lys residues present in Ub. Furthermore, the ART and PDE domains retain activity when translated as separate polypeptides.

The ubiquitination of host proteins by the SidE family does not appear to have amino acid sequence specificity surrounding the Ser residue. In vitro, SidE-family members are pleiotropic and can ubiquitinate several proteins. However, during Legionella infection, the reaction appears to be more specific for host proteins involved in the formation of the Legionella containing vacuole (LCV), a specialized organelle in which the bacterium replicates. Within the SidE-family genomic neighborhood lies the SidJ pseudokinase that polyglutamylates and inactivates the SidE effectors and DupA and DupB, which specifically deubiquitinate Ser-phosphoribose linkages introduced into host proteins by the SidE-family. Additionally, the Ser-phosphoribose Ub bond can be cleaved by hydroxylamine (NH₂OH).

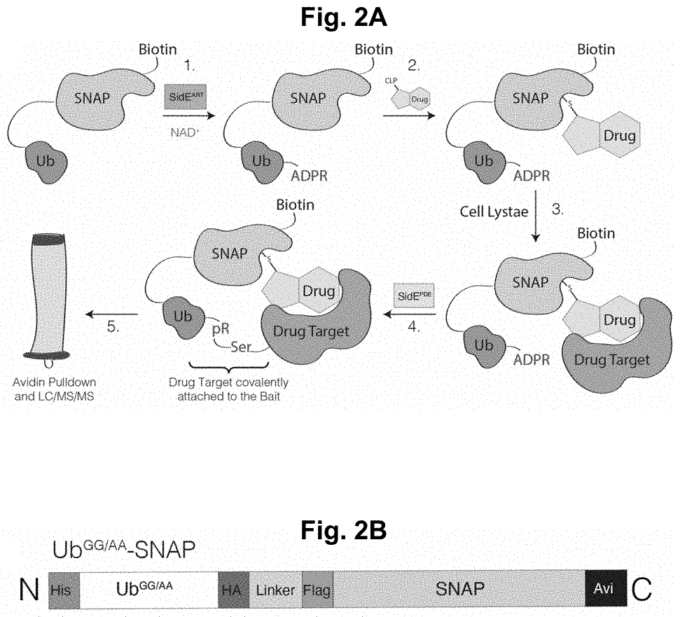

In this example, a biomolecule sensor was generated as part of a strategy to detect protein targets of small molecules. FIG. 2A shows an illustration of the strategy. In brief, a small molecule was displayed on a SNAP tag that was genetically fused to ADP-ribosylated ubiquitin. Protein targets which bind the small molecule were brought into proximity of the bait, then covalently linked to Ub Arg42 by exogenously added PDE domain. Labeled proteins can then be enriched by means of a biotin moiety preset in the bait fusion and identified by mass spectrometry.

First, a modified bacterial expression pProEx-1 vector containing a guanine insertion in the MCS (pProEx-2) was used to clone the human Ub coding sequence with the C-terminal diglycine mutated to dialanine (Ub^{GG/AA}), an HA-Tag, a GGGGTx3 linker, a FLAG-Tag, a SNAP-Tag, and finally a C-terminal Avi-Tag (Ub^{GG/AA}-SNAP). Specifically, Ub^{GG/AA} was fused to a SNAP-tag through a flexible linker and an Avi-tag for biotinylation. The SNAP tag was an engineered version of the DNA repair enzyme O6-alkylguanine-DNA alkyltransferase (AGT). AGT reacted specifically with derivatives of benzylguanine (BG) and chloropyrimidines (CLP) through an internal Cys residue leading to the covalent addition of the BG/CLP derivative to the SNAP tag. FIG. 2B shows an illustration of the Ub^{GG/AA}-SNAP construct. A C145A SNAP-Tag mutant of the above construction was also created by QuickChange site directed mutagenesis.

Next, the fusion proteins were expressed in E. coli and purified by Ni-NTA affinity chromatography from cell extracts. In brief, plasmids were transformed into Rosetta (BL21 derivative) competent cells and plated overnight. LB cultures were grown in the presence of Ampicillin (pProEx2) or Kanamycin (ppSumo) until the O.D. reached 0.6-1.0 and then induced with a final concentration of 0.4 mM IPTG overnight at room temperature. Cells were pelleted by centrifugation at 3000×g or 15 minutes and resuspended in 30 mL of a buffer containing 50 mM Tris-HCl pH 8, 150 mM NaCl, 1 mM DTT, and 1 mM PMSF. Cells were sonicated for a total process time of 2 minutes and 30 seconds on ice with 59 seconds rests in between 30 second pulses. Resulting lysates were centrifuged at 30,000×g for 30 minutes at 4° C. Cleared supernatants were incubated with 500 μL of washed Ni-NTA beads for 30 minutes ar 4° C. while rocking. Beads were washed with 30 mL of a buffer containing 50 mM Tris-HCl pH 8, 150 mM NaCl, 1 mM DTT, and 25 mM imidazole pH 8. Finally, proteins were eluted from the beads using the above buffer with 300 mM imidazole in a total volume of 5-10 mL.

In addition to the Ub^{GG/AA}-SNAP constructs, recombinant isolated ADP Ribosyltransferase (ART) domains and HD-like phosphodiesterase (PDE) domains were prepared. Briefly, the ART (residues 519-1100; SdeA^{ART}) and PDE domains (residues 178-652) coding sequences of the Legionella pneumophila effector SdeA were amplified from Legionella pneumophila Philadelphia-1 strain genomic DNA (gDNA) and cloned into a modified pet28a vector containing an N-terminal 6x-His-Sumo-tag (ppSumo). The coding sequence of the E. coli BirA was amplified from gDNA (Rosetta BL-21) and cloned into the ppSumo vector as well.

The recombinant isolated domains were expressed in E. coli and purified by Ni-NTA affinity chromatography from cell extracts as described above. Additionally, for proteins with a Sumo tag, 20 μL of 2 mg/mL Sumo protease, ULP was used to cleave the tag at 4° C. overnight. Resulting proteins were concentrated and loaded onto a superdex 200 or superdex 75 size exclusion column. Fractions corresponding to the appropriate proteins were pooled and frozen at −80° C. at 2-4 mg/mL.

Next, Ub^{GG/AA}-SNAP was ADP ribosylated by incubation with NAD+ and the ART domain of the SidE family member, SdeA (residues 519-1100; hereafter referred to as SdeA^{ART}). In brief, wild type Ub^{GG/AA} (WT) and C145A Ub^{GG/AA}-SNAP constructs were grown in 1 L cultures and purified using Ni-NTA beads as described above. In parallel, 500 mL cultures of E. coli BirA and SdeA^{ART} protein were expressed and purified. Directly following elution from the Ni-NTA beads, SdeA^{ART} and Ub^{GG/AA}-SNAP elution's were mixed and supplemented with 1 mM NAD+. The ADP-ribosylation reaction was incubated at room temperature for 1-2 hours before concentrating and loading onto a superdex 200 column. Fractions corresponding to Ub^{GG/AA}-SNAP (or C145A Ub^{GG/AA}-SNAP) were pooled and incubated with the E. coli BirA elution and supplemented with 5 mM MgCl₂, 2 mM ATP, and 1 mM biotin. 20 μL of 2 mg/mL ULP was also added to remove the 6xHis-Sumo tag from E. coli BirA. Reactions were incubated for 1-2 hours at room temperature. The proteins were buffer exchanged into imidazole-free buffer and incubated with Ni-NTA beads to separate the E. coli BirA from Ub^{GG/AA}-SNAP and C145A Ub^{GG/AA}-SNAP constructs. The resulting ADP ribosylated and biotinylated Ub^{GG/AA}-SNAP protein (WT and C145A) is hereafter referred to as SidBait. The Ub^{GG/AA}-SNAP protein (SidBait) was eluted in 300 mM imidazole, concentrated to 2 mL, and loaded onto the superdex 75 column. Fractions corresponding to SidBait were collected and frozen at 2 mg/mL until further use.

### Example 2. Characterization of SidBait Constructs

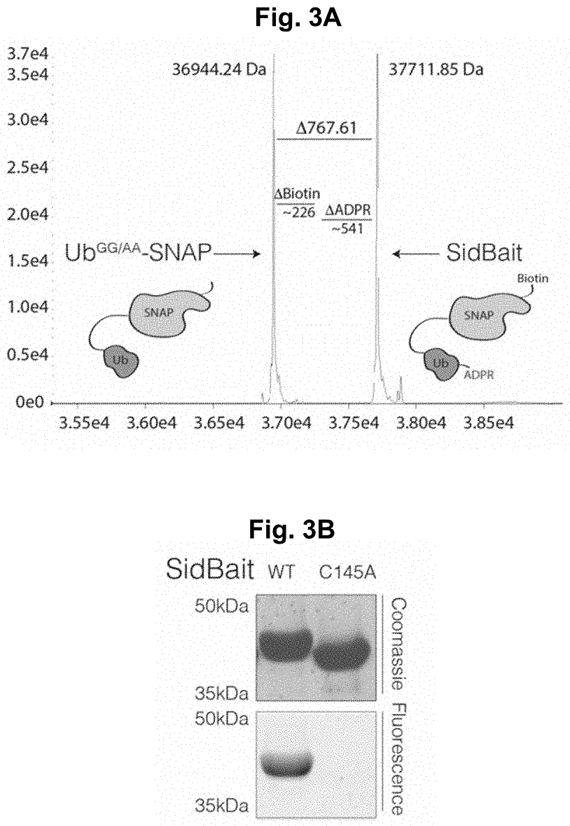

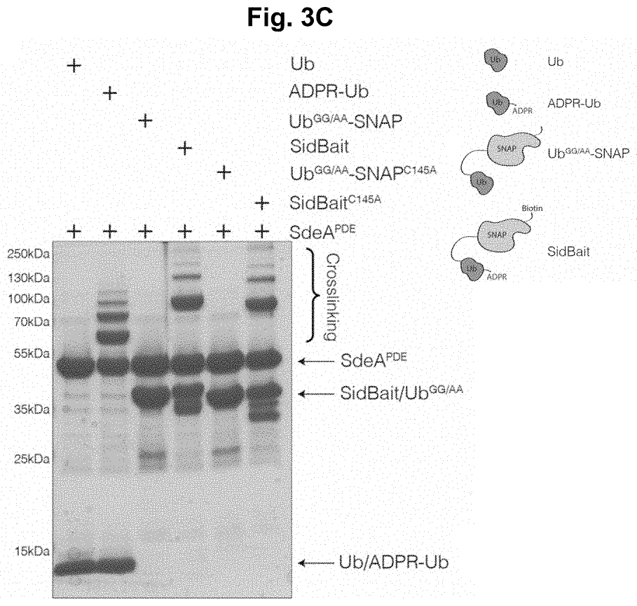

Intact mass analysis was next performed on SidBait. Briefly, protein samples (Ub^{GG/AA}-SNAP and Ub^{GG/AA}-SNAP treated with SdeA^{ART} and E. coli BirA to generate SidBait) were analyzed by LC/MS, using a Sciex X500B Q-TOF mass spectrometer coupled to an Agilent 1290 Infinity II HPLC. Samples were injected onto a POROS R1 reverse-phase column (2.1×30 mm, 20 μm particle size, 4000 Å pore size), desalted, and the amount of buffer B was manually increased stepwise until the protein eluted off the column. Buffer A contained 0.1% formic acid in water and buffer B contained 0.1% formic acid in acetonitrile. The mobile phase flow rate was 300 μL/min. The mass spectrometer was controlled by Sciex OS v.1.6.1 using the following settings: Ion source gas 1 30 psi, ion source gas 2 30 psi, curtain gas 35, CAD gas 7, temperature 300° C., spray voltage 5500 V, declustering potential 80 V, collision energy 10 V. Data was acquired from 400-2000 Da with a 0.5 second accumulation time and 4 time bins summed. The acquired mass spectra for the proteins of interest were deconvoluted using BioPharmaView v. 3.0.1 software (Sciex) in order to obtain the molecular weights. The peak threshold was set to ≥5%, reconstruction processing was set to 10 iterations with a signal-to-noise threshold of ≥5 and a resolution of 20000. Intact mass analysis confirmed the stoichiometric modification of SidBait with one ADP ribose and one biotin (FIG. 3A). Also, when the two spectra were overlaid on the same graph, FIG. 3A depicts the increased mass of ˜767.61 Da, which corresponded to the addition of an ADP-ribose (˜541 Da) and a biotin (˜226 Da) on SidBait compared to Ub^{GG/AA}-SNAP.

To determine whether the SNAP tag on SidBait was functional, SNAP-Cell Oregon Green (a fluorescent dye, which contains a BG and can be conjugated to SNAP tags) was incubated with WT or the C145A mutant of SidBait. The incorporation of the SNAP-Cell Oregon green dye (NEB S9104) was carried out in 50 mM Tris-HCl and 1 mM DTT with a final volume of 20 μL. 3 μg of WT and C145A SidBait constructs were incubated with 10 μM final concentration of the dye at room temperature for 1 hour. 5 μL of 5×SDS Loading Dye was added and samples were boiled for 5 minutes. 20 μL was loaded onto a 12% SDS-PAGE gel and stained with Coomassie (FIG. 3B, top panel). Gels were imaged in a BioRad Chemidoc MP using the green channel. When the reaction products were separated the by SDS PAGE, fluorescent modification was observed for the WT SidBait but not the C145A mutant (FIG. 3B, bottom panel). These results indicated that the SNAP tag in SidBait could be conjugated with a small molecule BG/CLP derivative.

To test whether the Ub^{GG/AA} domain of SidBait was a substrate for the PDE domain of SdeA and could be conjugated to proteins, Ub^{GG/AA}-SNAP and SidBait were incubated with the PDE domain of SdeA (residues 178-652, hereafter referred to as SdeA^{PDE}). The incubations, or SdeA^{PDE} crosslinking reactions, were carried out in 50 mM Tris-HCl pH 7 with a final volume of 20 μL. 6 μg of Ub^{GG/AA}-SNAP constructs or Ub alone (control) were incubated with 6 μg of SdeA^{PDE} domain at room temperature for 1 hour. 5 μL of 5×SDS loading dye was added to the samples and boiled for 5 minutes. 20 μL were loaded onto a 12% SDS-PAGE gel and after separation, were stained with Coomassie Blue for visualization. In the absence of a substrate, SdeA undergoes auto-ubiquitination detectable by laddering on SDS-PAGE. Once the reaction products were separated by SDS PAGE, crosslinking was observed for both WT and the C145A SidBaits but not the non-ADP-ribosylated Ub^{GG/AA}-SNAP (FIG. 3C). Collectively, these results indicated that SidBait and SidBait^{C145A} are functional and can be covalently crosslinked to proteins via Ser-phosphoribose-Ub bonds.

### Example 3. Use of SidBait Constructs to Detect Protein Targets

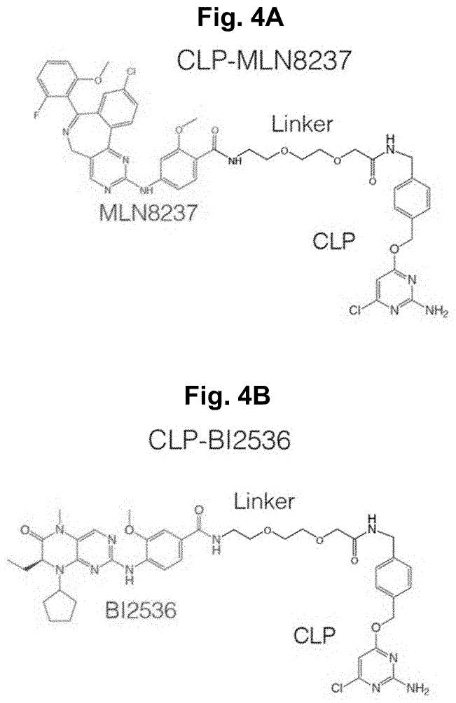

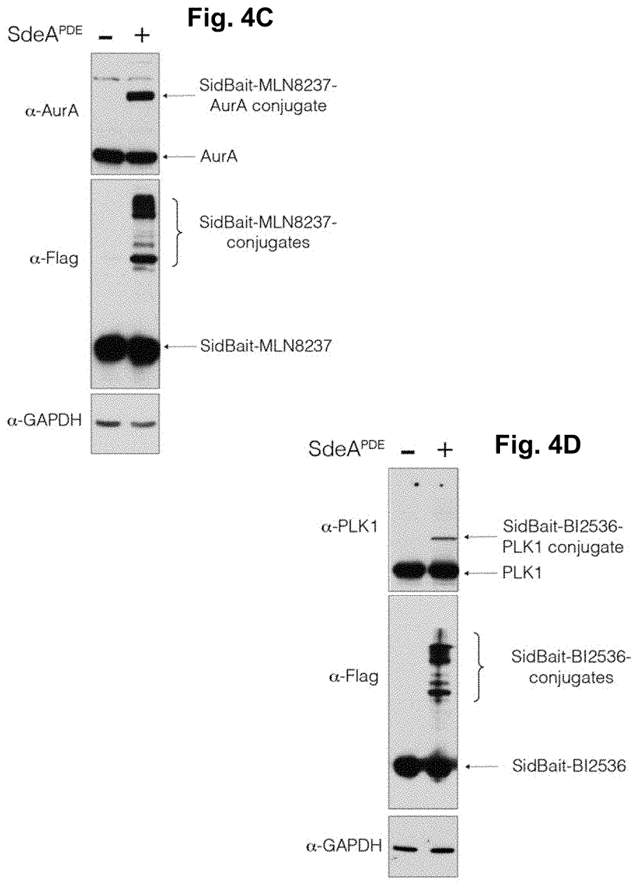

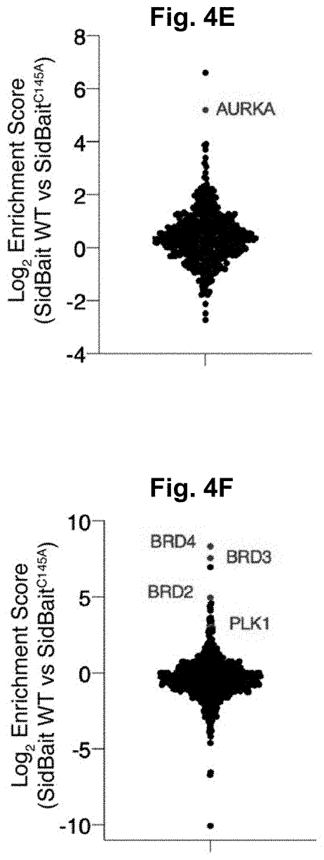

To test the use of SidBait constructs in the strategy depicted in FIG. 2A, chloropyrimidine (CLP) derivatives of the AurA kinase inhibitor MLN8237 (CLP-MLN8237; FIG. 4A) and the dual PLK1/BRD4 inhibitor BI2536 (CLP-BI2536; FIG. 4B) were first synthesized. AurA and PLK1 are potential targets for novel cancer therapeutics, and MLN8237 is actively pursued in oncology clinical trials. CLP-MLN8237 and CLP-BI2536 are both functional inhibitors of AurA and PLK1 in vitro and in cells. CLP-MLN8237 and CLP-BI2536 were synthesized according to methods similar to that of Bucko et al., eLife (2019) December 24; 8:e52220, hereby incorporated by reference in its entirety. Conjugations were carried out in 1×TBS (50 mM Tris-HCl pH 7.5, 150 mM NaCl) supplemented with 2 mM DTT at a final volume of 20 μL. 1.2 μL of 1 mM CLP-tagged drug was incubated with 18 μL of 2 mg/mL SidBait at room temperature for 1-2 hours. Final concentration was 50-60 μM for SidBait-MLN8237 and SidBait-BI2536.

Next, 300 μL of a concentrated HEK293A (human embryonic kidney 293 cell) lysate (˜3 mg/mL total protein) were added to each of the SidBait conjugation reactions. Following incubation on ice for 30-60 minutes, 60 μg of PDE domain (SdeA^{PDE}) was added and incubated at 30° C. for 2 hours. 50 μL of washed Streptavidin beads were added to the reaction and incubated overnight. Beads were then spun at 800×g for 1 minute at 4° C. Beads were washed once with 2% SDS, once with wash buffer 1 (50 mM Hepes pH 7, 0.1% Sodium deoxycholate, 1% Triton X-100, 1 mM EDTA, 500 mM NaCl), and once with wash buffer 2 (10 mM Tris-HCl pH 7, 0.5% Sodium deoxycholate, 0.5% NP-40, 1 mM EDTA, 250 mM LiCl). Following addition of SdeA^{PDE}, reaction products were separated by SDS PAGE along with a control (SidBait conjugation reactions collected before SdeA^{PDE}). Gels were subjected to protein immunoblotting for AurA (FIG. 4C) and PLK1 (FIG. 4D). Immunoblots showed formation of higher molecular weight species of endogenous AurA (FIG. 4C) and PLK1 (FIG. 4D) when incubated with SidBait in the presence of SdeA^{PDE}. These results suggested that AurA and PLK1 had successfully been crosslinked to their respective SidBaits.

To determine whether the SidBait approach is capable of identifying the correct targets of small molecules in an unbiased manner, semi-quantitative mass spectrometry was used to compare avidin pulldowns from HEK293A lysates that were incubated with WT or the C145A SidBait derivatives and subsequently treated with SdeA^{PDE}. In brief, proteins were digested on-beads prior to LC-MS/MS analysis. Proteins were first reduced with DTT (1 hr, 56° C.) and alkylated with iodoacetamine (45 min, 25° C. in the dark). Proteins were digested on-beads with sequencing grade trypsin (overnight, 37° C.). The next day, tryptic peptides were acidified with 5% trifluoroacetic acid and de-salted via solid phase extraction (SPE). LC-MS/MS experiments were performed on a Thermo Scientific EASY-nLC liquid chromatography system coupled to a Thermo Scientific Orbitrap Fusion Lumos mass spectrometer. To generate MS/MS spectra, MS1 spectra were first acquired in the Orbitrap mass analyzer (resolution 120,000). Peptide precursor ions were then isolated and fragmented using high-energy collision-induced dissociation (HCD). The resulting MS/MS fragmentation spectra were acquired in the ion trap. Label-free quantitative searches were performed using Proteome Discoverer 2.1 software (Thermo Scientific). Samples were searched against entries included in the Human Uniprot database. Search parameters included setting Carbamidomethylation of cysteine residues (+57.021 Da) as a static modification and oxidation of methionine (+15.995 Da) and acetylation of peptide N-termini (+42.011 Da) as dynamic modifications. Precursor and product ion mass tolerances of 10 ppm and 0.6 Da were used, respectively. Peptide spectral matches were adjusted to a 1% false discovery rate (FDR) and additionally proteins were filtered to a 5% FDR. Proteins were quantified by comparing area values determined via label-free quantitation using Proteome Discoverer software.

For the mass spectrometry analysis, a comparative table of WT vs C145A samples were obtained from the Mascot search. Proteins with less than 5 peptides were discarded. Samples missing scores in one of the samples were automatically assigned the average of the bottom 100 scores. Enrichment scores were calculated using the log₂ of the WT score divided by the C145A score. Gene names and enrichment scores were plotted using Prism software.

In the SidBait-MLN8237 experiments, AurA returned as the second highest enriched protein (FIG. 4E). Likewise, in the SidBait-1312536 pulldowns, PLK1 and the BRD family members, BRD2/3/4 were among the most enriched interactors (FIG. 4F). Collectively, these results suggested that SidBait is a robust, versatile and high-fidelity system for identifying protein targets of small molecules.

### Conclusion to Examples 1-3

Many drugs that score well in phenotypic screens of disease models are often abandoned because of toxicity due to off-target effects and high dosage requirements. Exemplary examples 1-3 showed that SidBait is an unbiased, proximity ligation labelling method for identifying drug targets with low background and no requirement for a priori knowledge. Fast and modular target and off-target identification of these drugs would allow for modification and optimization of lead compounds to produce clinically efficacious and safe drugs. Thus, SidBait can revolutionize drug discovery by providing a reliable, simple and cost-effective pipeline for target identification. As such, SidBait can bolster the number of new drugs introduced into clinical trials and transform the way pharmaceutical companies approach drug discovery.

What is claimed is:

1. A biomolecule sensor comprising three constructs, the three constructs which together have the formula (I):
Ub-T1-L-T2-S-A;
R; and
P (I)
wherein:
Ub is a peptide having a genetically modified sequence for ubiquitin;
T1 and T2 are each optional epitope tags;
L is a linker;
S is a self-labeling protein tag;
A is biotin-acceptor peptide;
R is a peptide comprising a ADP-Ribosyltransferase (ART) domain of a SidE-ligase protein, at least one ortholog of SidE-ligase protein, or at least one paralog of a SidE-ligase protein; and
P is a peptide comprising a phosphodiesterase (PDE) domain of a SidE-ligase protein, at least one ortholog of SidE-ligase protein, or at least one paralog of a SidE-ligase protein.

2. The biomolecule sensor of claim 1, wherein Ub comprises at least one genetic modification in the C-terminal peptide region.

3. The biomolecule sensor of claim 1, wherein R and/or P comprises a peptide comprising a fragment of at least one paralog of a SidE effector protein from a species of Legionella.

4. The biomolecule sensor of claim 3, wherein R and/or P comprises a peptide comprising a fragment of Legionella pneumophila effector SdeA.

5. The biomolecule sensor of claim 1, wherein T1 and/or T2 comprises one or more epitope tags selected from a group consisting of ALFA-tags, AviTags, C-tags, Calmodulin-tags, polyglutamate tags, polyarginine tags, E-tags, FLAG-tags, HA-tags, His-tags, Myc-tags, NE-tags, Rho1D4-tags, S-tags, SBP-tags, Softag 1s, Softag 3s, Spot-tags, Strep-tags, T7-tags, TC tags, Ty tags, V5 tags, VSV-tags, and Xpress tags.

6. The biomolecule sensor of claim 1, wherein L comprises a peptide having 2 to 20 amino acids.

7. The biomolecule sensor of claim 1, wherein S comprises one or more self-labeling protein tags selected from a group consisting of Halo-tags, SNAP-tags, CLIP-tags, ACP-tags, MCP-tags, and UV-cross-linking capable tags.

8. The biomolecule sensor of claim 7, wherein the UV-cross-linking capable tags comprise aryl azides, fluorinated aryl azides, azido-methyl-coumarins, benzophenones, anthraquinones, diazo compounds, diazirines, psoralens, 5-halo-uridines, 5-halo-cytosines, 7-halo-adenosines, 2-nitro-5-azidobenzoyls, fluorinated aryl azides, amino-benzophenones, or derivatives thereof.

9. The biomolecule sensor of claim 1, wherein A comprises one or more biotin-acceptor peptides comprising 10 to 400 amino acids.

10. A system for proximity-based labeling of a biomolecule, the system comprising:
(a) at least one construct comprising a genetically modified ubiquitin (Ub), a self-labeling protein tag, a linker, or a combination thereof;
(b) a biomolecule derivative; and
(c) at least two protein domains of a SidE-ligase protein, an ortholog of SidE-ligase protein, or a paralog of a SidE-ligase protein.

11. The system of claim 10, wherein the at least two protein domains of SidE comprise at least one full-length SidE-ligase protein, an ortholog of a full-length SidE-ligase protein, or a paralog of a full-length SidE-ligase protein.

12. The system of claim 10, wherein the at least two protein domains of SidE comprise at least one isolated protein domain of SidE-ligase protein, isolated protein domain of an ortholog of SidE-ligase protein, or isolated protein domain of a paralog of a SidE-ligase protein.

13. The system of claim 10, wherein one of the at least two protein domains of a SidE-ligase protein, an ortholog of SidE-ligase protein, or a paralog of a SidE-ligase protein comprises an ADP-Ribosyltransferase (ART) domain.

14. The system of claim 10, wherein one of the at least two protein domains of a SidE-ligase protein, an ortholog of SidE-ligase protein, or a paralog of a SidE-ligase protein comprises a phosphodiesterase (PDE) domain.

15. The system of claim 10, wherein one of the at least two protein domains of a SidE-ligase protein comprising a peptide fragment of Legionella pneumophila effector SdeA.

16. The system of claim 10, further comprising at least one epitope tag, at least one biotin-acceptor peptide, or any combination thereof.

17. A method for detecting a protein interaction with a target biomolecule, the method comprising:
(a) contacting a sample comprising the target biomolecule with a biomolecule sensor, the biomolecule sensor comprising three constructs, the three constructs which together have the formula (I):
Ub-T1-L-T2-S-A;
R; and
P (I)
wherein:
Ub is a peptide having a genetically modified sequence for ubiquitin;
T1 and T2 are each optional epitope tags;
L is a linker;
S is a self-labeling protein tag or a target protein;
A is biotin-acceptor peptide;
R is a peptide comprising a ADP-Ribosyltransferase (ART) domain of a SidE-ligase protein, at least one ortholog of SidE-ligase protein, or at least one paralog of a SidE-ligase protein; and
P is a peptide comprising a phosphodiesterase (PDE) domain of a SidE-ligase protein, at least one ortholog of SidE-ligase protein, or at least one paralog of a SidE-ligase protein;
(b) detecting whether the target biomolecule is bound to S, indicating that the protein interaction with a target biomolecule is present in the sample.

18. The method of claim 17, wherein the target biomolecule comprises a derivative of the target biomolecule comprising at least one ultraviolet cross linking side group, at least one chemical handle, or any combination thereof.

19. The method of claim 17, wherein the target biomolecule is bound to S by a phosphoribose linkage.

20. The method of claim 17, wherein Ub is ADP-ribosylated by R.
